SEQUENCE LISTING (45 sequences)

SEQ ID NO: 1 - protein, 23 aa, Xenopus laevis
GIGKFLHSAKKFGKAFVGEIMNS

SEQ ID NO: 2 - protein, 21 aa, Xenpus laevis
  misc_feature: Leu at position 21 is amide
GMASKAGGIAGKIAKVALKAL

SEQ ID NO: 3 - protein, 21 aa, Artificial Sequence
  artificial mutant
KLASKAGKIAGKIAKVALKAL

SEQ ID NO: 4 - protein, 21 aa, Artificial Sequence
  artificial mutant
KIAGKIAKIAGKIAKIAGKIA

SEQ ID NO: 5 - protein, 21 aa, Artificial Sequence
  artificial mutant
KIAGKIAKWAGKIAKIAGKIA

SEQ ID NO: 6 - protein, 21 aa, Artificial Sequence
  artificial mutant
KLAGLAKKLAGLAKKLAGLAK

SEQ ID NO: 7 - protein, 21 aa, Artificial Sequence
  artificial mutant
KLAGLAKKWAGLAKKLAGLAK

SEQ ID NO: 8 - protein, 18 aa, Artificial Sequence
  artificial mutant
KIGAKIKIGAKIKIGAKI

SEQ ID NO: 9 - protein, 18 aa, Artificial Sequence
  artificial mutant
KIGAKIKWGAKIKIGAKI

SEQ ID NO: 10 - protein, 18 aa, Artificial Sequence
  artificial mutant
KKIGAIKKIGAIKKIGAI

SEQ ID NO: 11 - protein, 18 aa, Artificial Sequence
  artificial mutant
KIKGAIKIKGAIKIKGAI

SEQ ID NO: 12 - protein, 18 aa, Artificial Sequence
  artificial mutant
KIGKAIKIGKAIKIGKAI

SEQ ID NO: 13 - protein, 18 aa, Artificial Sequence
  artificial mutant
KIGAIKKIGAIKKIGAIK

SEQ ID NO: 14 - protein, 18 aa, Artificial Sequence
  artificial mutant
KLAGKLAKLAGKLAKLAG

SEQ ID NO: 15 - protein, 21 aa, Artificial Sequence
  artificial mutant
KKLAGLAKKLAGLAKKLAGLA

SEQ ID NO: 16 - protein, 21 aa, Artificial Sequence
  artificial mutant
KLKAGLAKLKAGLAKLKAGLA

SEQ ID NO: 17 - protein, 21 aa, Artificial Sequence
  artificial mutant
KLAKGLAKLAKGLAKLAKGLA

SEQ ID NO: 18 - protein, 21 aa, Artificial Sequence
  artificial mutant
KLAGKLAKLAGKLAKLAGKLA

SEQ ID NO: 19 - protein, 21 aa, Artificial Sequence
  artificial mutant
KLAGLKAKLAGLKAKLAGLKA

SEQ ID NO: 20 - protein, 21 aa, Artificial Sequence
  artificial mutant
KIGAKIKIGAKIKIGAKIKIG

SEQ ID NO: 21 - protein, 22 aa, Artificial Sequence
  artificial mutant
GIGKFLKKAKKFGKAFVKILKK

SEQ ID NO: 22 - protein, 4 aa, Artificial Sequence
  artificial mutant
SVKV

SEQ ID NO: 23 - protein, 10 aa, Artificial Sequence
  artificial mutant
FKVKFKVKVK

SEQ ID NO: 24 - protein, 15 aa, Artificial Sequence
  artificial mutant
KLKLKLKLKLKLKLK

SEQ ID NO: 25 - protein, 18 aa, Artificial Sequence
  artificial mutant
KKIGAIKKWGAIKKIGAI

SEQ ID NO: 26 - protein, 18 aa, Artificial Sequence
  artificial mutant
KIKGAIKWKGAIKIKGAI

SEQ ID NO: 27 - protein, 18 aa, Artificial Sequence
  artificial mutant
KIGKAIKWGKAIKIGKAI

SEQ ID NO: 28 - protein, 18 aa, Artificial Sequence
  artificial mutant
PRIIRRIIRIIRRIIRRI

SEQ ID NO: 29 - protein, 18 aa, Artificial Sequence
  artificial mutant
KIGAKIKWGAKIKIGAKI

SEQ ID NO: 30 - protein, 18 aa, Artificial Sequence
  artificial mutant
KIGAIKKWGAIKKIGAIK

SEQ ID NO: 31 - protein, 21 aa, Artificial Sequence
  artificial mutant
KKLAGLAKKWAGLAKKLAGLA

SEQ ID NO: 32 - protein, 21 aa, Artificial Sequence
  artificial mutant
KLKAGLAKWKAGLAKLKAGLA

SEQ ID NO: 33 - protein, 21 aa, Artificial Sequence
  artificial mutant
KLAKGLAKWAKGLAKLAKGLA

SEQ ID NO: 34 - protein, 21 aa, Artificial Sequence
  artificial mutant
KLAGKLAKWAGKLAKLAGKLA

SEQ ID NO: 35 - protein, 21 aa, Artificial Sequence
  artificial mutant
KLAGLKAKWAGLKAKLAGLKA

SEQ ID NO: 36 - protein, 21 aa, Artificial Sequence
  artificial mutant
KLAGLAKKWAGLAKKLAGLAK

SEQ ID NO: 37 - protein, 18 aa, Artificial Sequence
  artificial mutant
KIGAKIKIGAKIKWGAKI

SEQ ID NO: 38 - protein, 12 aa, Artificial Sequence
  artificial mutant
KIGAKIKWGAKI

SEQ ID NO: 39 - protein, 6 aa, Artificial Sequence
  artificial mutant
KWGAKI

SEQ ID NO: 40 - protein, 11 aa, Artificial Sequence
  artificial mutant
KLKWKLKLKLK

SEQ ID NO: 41 - protein, 18 aa, Artificial Sequence
  artificial mutant
RIRIRIRIRIRIRIRIRI

SEQ ID NO: 42 - protein, 18 aa, Artificial Sequence
  artificial mutant
KXGXKXKXGXKXKXGXKX

SEQ ID NO: 43 - protein, 18 aa, Artificial Sequence
  artificial mutant
KIGAKSKHGAKIKIGAKI

SEQ ID NO: 44 - protein, 14 aa, Artificial Sequence
  artificial mutant
KLKLKLKWKLKLKL

SEQ ID NO: 45 - protein, 18 aa, Artificial Sequence
  artificial mutant
KLKLKLKWKLKLKLKLKL

(19) United States
(12) Patent Application Publication
(10) Pub. No.: US 2004/0249122 A1
(43) Pub. Date: Dec. 9, 2004

(54) Cationic, amphipathic beta-sheet peptides and uses thereof

(76) Inventor: John F. Blazyk, Athens, GA (US)

Correspondence Address: BROWDY AND NEIMARK, P.L.L.C., 624 NINTH STREET, NW, SUITE 300, WASHINGTON, DC, 20001-5303, US

(21) Appl. No.: 10/181,898

(22) PCT Filed: Feb. 15, 2001
(86) PCT No.: PCT/US01/04822

(51) Int. Cl. C07K 7/08; C07K 7/06

(52) U.S. Cl. 530/324; 530/325; 530/326; 530/327; 530/328; 530/329

(57) ABSTRACT

This invention relates to an antimicrobial compound which is (a) a peptide having a length of 8-50 amino acids, a net charge of at least four, a hydrophobic moment as a beta sheet which is at least 0.2 higher than its hydrophobic moment as an alpha helix, and having detectable membrane disrupting activity against at least one microbial pathogen, and substantially no membrane disrupting activity against mammalian cells, or (b) a peptoid, peptidomimetic or nonpeptidic analogue of a peptide according to (a) above. And to antimicrobial use thereof.

This application is a nonprovisional of Serial No. 60/182,495 filed Feb. 15, 2000, which is hereby incorporated by reference.

### MENTION OF GOVERNMENT GRANT

The invention disclosed herein was developed with the assistance of NIH Grant 1-R-15-AI-47165. The U.S. Government has certain rights in the invention.

### BACKGROUND OF THE INVENTION

1. Field of the Invention

This invention relates to the use of cationic peptides which can assume an amphipathic beta-sheet secondary structure for clinical and diagnostic purposes, especially inhibition of microbial activity.

2. Description of the Background Art

We know that diseases caused by bacteria have afflicted humans since the beginning of recorded history. The Black Plague devastated the population of Europe in the fourteenth century. Other major outbreaks of bacterial infection wreaked havoc on a regular basis through the nineteenth century. It was not until the mid-1800's that scientists such as Louis Pasteur first linked microorganisms with human disease. This breakthrough set the stage for the identification of therapeutic agents that could selectively kill them.

In the early 1900's, a compound named Salvarsan, an arsenic-containing dye, was developed by Paul Erhlich as a treatment for syphilis. Soon thereafter, in the 1920's, Alexander Fleming discovered lysozyme (present in human tears) and penicillin; however, the clinical application of these discoveries did not materialize until the 1940's. In the meantime, a new dye called Prontosil was found to cure Streptococcus infections in mice. This compound, a sulfonamide, opened the door to the development of other antibiotics. In the early 1940's, Howard Florey and his coworkers finally purified penicillin. Following the fire at the Cocoanut Grove in Boston in 1942 (where crude penicillin used to treat burn victims received tremendous acclaim), pharmaceutical companies dramatically increased the production of penicillin, leading to its widespread availability and use. Penicillin was declared a “miracle drug”. In the late 1940's and early 1950's, oral penicillin was available over-the-counter without a prescription.

Since most living organisms are very similar at the molecular level, it is difficult to find substances that are lethal to certain organisms without being harmful to others. Antibiotics have proven effective in eliminating or at least greatly reducing the incidence of many clinical problems caused by bacteria because these compounds possess the necessary selectivity to attack bacterial cells while sparing human cells. These antibiotics rely upon differences between these cells. For instance, penicillin is effective because it targets the bacterial cell wall, for which there is no similar counterpart in human cells. Unfortunately, the widespread use of common antibiotics such as penicillin has selected for resistant strains that are no longer susceptible to these agents.

In 1945, Fleming predicted that improper use and overuse of the new drug would lead to the development of resistant microorganisms. Penicillin resistance soon materialized; however, the discovery of new antibiotics lessened the impact of the resistance problem. Streptomycin and other aminoglycosides, chloramphenicol, tetracyclines, cephalosporins, quinolones, semi-synthetic penicillins like ampicillin and methicillin, and super-potent drugs like vancomycin enriched the antimicrobial arsenal. In the past two decades, only a small number of new antimicrobials have appeared on the market. The array of available drugs seemed to be sufficiently vast to stifle interest in costly new development programs.⁷

Over the years, however, more and more microorganisms, exposed to more and more antibiotics, have adapted to these compounds. Resistance to antimicrobial drugs is now a worldwide problem. The emergence of methicillin-resistant Staphylococcus aureus in the 1970's was a major setback in antibiotic therapy. The prospect of vancomycin-resistant S. aureus looms on the horizon. Multiply-drug-resistant strains of Mycobacterium tuberculosis have resulted from improper or incomplete therapy. Outbreaks of tuberculosis are now commonplace among indigent populations in cities throughout the U.S. Since resistance is appearing to even the most potent antibiotics such as vancomycin, the development of new approaches in antimicrobial therapy is imperative.¹

Some new ideas include limiting the ability of microorganisms to transfer plasmids containing resistance genes, reducing the virulence of disease-causing organisms, and looking for new models of antimicrobial compounds.⁸⁻¹⁰

The host defense systems of animals are potential sources of new ideas in antimicrobial therapy. In particular, small cationic peptides represent a large class of weaponry used in the protection against bacterial infection by animals.^{11, 12} A diverse collection of host defense peptides, discovered in a wide range of species, shares the common characteristics of a net positive charge and the ability to form amphipathic structures. Many of these peptides appear to exert their protective effect by permeabilizing the membranes of target organisms. The efficacy of these peptides results from their ability to disrupt prokaryotic membranes at concentrations that are not harmful to host membranes. Frog skin is a particularly rich source of antimicrobial peptides, including magainins and PGLa.

The discovery of naturally occurring antimicrobial peptides opened a new pathway for antibiotic development.^{2, 3} Magainin 1 and magainin 2, first isolated from frog skin in 1987, are representative of the class of small linear cationic peptides that can kill both Gram-positive and Gram-negative bacteria by increasing the permeability of the plasma membrane at concentrations that do not induce hemolysis.^{4, 5} PGLa, also isolated from frog skin, has greater antimicrobial activity than magainins while retaining low hemolytic activity.⁶ A common feature of these peptides is their capacity to form an amphipathic α-helix (with polar and nonpolar groups on opposite faces of the helix), a structural feature believed to be important in their function as antimicrobial agents.

These linear cationic peptides, containing 21-23 amino acid residues, demonstrate broad antimicrobial activity; however, relatively high concentrations are necessary to kill most target organisms. It is possible to enhance antimicrobial activity through simple modifications of the native peptides. For instance, substituting Ala for Glu-19 in magainin 2 amide-substantially increases antimicrobial activity (47, 52).

Our earliest work involved looking at the role of the outer membrane and LPS in the interaction between magainin 2 and the Gram-negative cell envelope. ^{16-18, 30, 31} Magainin 2 altered the thermotropic properties of the outer membrane-peptidoglycan complexes from wild-type Salmonella typhimurium and a series of LPS mutants that display differential susceptibility to the bactericidal activity of cationic antibiotics. These results were correlated with the LPS phosphorylation pattern and charge (characterized by high-resolution ³¹P NMR) and outer membrane lipid composition, and were compared to the bactericidal susceptibility. LPS mutants showed a progressive loss of resistance to killing by magainin 2 as the length of the LPS polysaccharide moiety decreased. Disordering of the outer membrane lipid fatty acyl chains by magainin 2 depended primarily upon the magnitude of LPS charge rather than the length of the LPS polysaccharide. While disruption of outer membrane structure most likely is not the primary factor leading to cell death, the susceptibility of Gram-negative cells to magainin 2 is associated with factors that facilitate the transport of the peptide across the outer membrane, such as the magnitude and location of LPS charge, the concentration of LPS in the outer membrane, outer membrane molecular architecture, and the presence or absence of the O-antigen side chain.

Magainins and PGLa function by binding to bacteria and inducing leakage. The selectivity for bacteria over mammalian cells is based at least in part on the presence of anionic lipids on the outer surface of bacteria, such as lipopolysaccharide (LPS) in the outer membrane of Gram negative organisms¹⁶⁻¹⁸ and phosphatidylglycerol (PG) and diphosphatidylglycerol (DPG) in the plasma membrane.¹⁹ In contrast, the outer surface of mammalian cells is populated almost exclusively by zwitterionic phospholipids, primarily phosphatidylcholine (PC) and sphingomyelin. Magainin-induced leakage in large unilamellar vesicles (LUV) is generally favored as the ratio of acidic to neutral lipids increases. Thus, electrostatic interactions play an integral role in at least the initial binding process.

Magainins and related peptides can adopt some degree of α-helical structure in the presence of TFE or when bound to lipid bilayers. In aqueous solution, however, they possess no discernable secondary structure. While there is general agreement that an α-helix is the dominant conformation, there is debate concerning the level of helicity, ranging between 60 and 90%.²⁰ In spite of the experimental data, most often these peptides are considered to be entirely helical. This point may be important in evaluating models of how the peptides exert their lytic effects. Another area of contention is the orientation of the peptides with respect to the plane of the lipid bilayer. Peptide orientation has been evaluated using solid-state NMR,^{21, 22} fluorescence quenching,²³ and oriented circular dichroism (CD).²⁴ NMR and fluorescence results show that the peptides remain associated with the lipid polar groups; however, the CD study suggests that the helical axis of the peptide changes from parallel to the lipid bilayer to perpendicular when the peptide-to-lipid ratio exceeds 1:30.

Much attention has been focused on whether these peptides form channels or pores in lipid bilayers and membranes, and if so, the exact nature of these pores. An early model proposed that the peptides, after binding to the surface of the membrane, self-associate to form a multimeric bundle that inserts across the bilayer, with the polar helical faces lining the cavity of the pore.^{3, 25} Other more complex models were developed that include lipid molecules as part of the pore.²⁶ More recently, a model invoking a supramolecular organization of lipids and peptides forming a torus was proposed by Matsuzaki²⁷ and Huang.²³ If these models are correct, there are no direct peptide-peptide interactions involved in pore formation. In fact, the pores might simply be membrane defects similar to those that would result from the accumulation of detergent molecules in the bilayer on the way to formation of micelles and the loss of bilayer structure. The peptides binding to the interfacial region of the lipid bilayer may well mimic the action of detergents by expanding the surface area to induce sufficient positive curvature to destabilize the bilayer.

Thus far, it appears that microorganisms have not been able to develop resistance to magainins and related peptides, even after chronic exposure to sublethal doses. This is probably due to the fact that the peptides do not bind to a specific receptor, but instead are attracted tQ the anionic membrane surface that would be difficult for the organism to alter extensively. The most likely resistance mechanism against these molecules is the presence of proteases that could degrade the peptides. The emergence of proteases might have less impact if it is possible to identify many diverse sequences that possess potent antimicrobial activity and good selectivity for bacterial cells.

In 1987, Zasloff isolated magainin 1 and magainin 2, a pair of 23-residue peptides with broad spectrum antimicrobial activity.⁴ These peptides kill microorganisms by increasing the permeability of the bacterial plasma membrane at concentrations that are not hemolytic. The sequence of magainin 2 is shown below:

| GIGKFLHSAKKFGKAFVGEIMNS | (SEQ ID NO:1) |
|---|---|
| 1 5 10 15 20 | |

In water, this peptide has no discernable secondary structure, but in the presence of trifluoroethanol (TFE), it is mostly α-helical ¹³. Due to the placement of the polar and nonpolar residues in the amino acid sequence, the resulting α-helix is reasonably amphipathic.

Magainin 2 amide exhibits only weak potency (i.e., its minimum inhibitory concentration (MIC) values against most microorganisms is =256 μg/mL). Deletion of E at position 19 or its replacement by A greatly increases the antimicrobial activity of the peptide (see Table 2).¹⁵ These analogues are more cationic (+4 vs. +3), but their amphipathic character as α-helices is virtually unchanged. We have studied the conformation and activity of the E19A analogue.

MSI-78, with a net charge of +10, is a peptide derived from the E19 deletion analogue of magainin 2 amide that is under development as a topical antimicrobial agent by Magainin Pharmaceuticals, Inc.¹²

Its sequence is:

| MSI-78 | GIGKFLKKAKKFGKAFVKILKK- NH₂ | (SEQ ID NO:21) |
|---|---|---|
| | 1 5 10 15 20 | |

PGLa is a similar peptide also discovered in frog skin.⁶ Like magainins, it is a small cationic peptide with the potential to form an amphipathic α-helix. PGLa is substantially more active than magainin 1 or 2, while maintaining low hemolytic activity. Its amino acid sequence is:

| GMASKAGGIAGKIAKVALKAL- NH₂ | (SEQ ID NO:2) |
|---|---|
| 1 5 10 15 20 | |

A more potent analogue was produced by replacing two glycines with lysines (G1K, G8K). In this analogue, there are three heptamers with the sequence KXXXKXX. Three new peptides (each derived from one of the heptamers) were made as trimeric repeats. The peptide from the middle heptamer, (KIAGKIA)₃-NH₂ (SEQ ID NO:4), possessed the most potent antimicrobial activity, on a par with MSI-78, even though its net charge is much less (see Table 2).¹¹

Numerous analogues with sequences derived from these peptides have been prepared and examined. In all cases of which applicant(s) are aware, the strategy employed in enhancing activity involved increasing the amphipathic α-helical character of the peptide.

Several other linear amphipathic β-sheet peptides have been examined previously. An 18-residue KL repeat was reported to have no appreciable antimicrobial or hemolytic activity (52). Peptides containing 6-12 residues with repeats of either SVKV (SEQ ID NO:22) or KV were shown to adopt a β-sheet structure in the presence of lipid(23) While some of these peptides could induce leakage in lipid vesicles, none were antimicrobial below a concentration of 100 μg/mL. The peptide FKVKFKVKVK (SEQ ID NO:23) was able to inhibit the growth of E. coli, S. aureus, and P. aeruginosa at concentrations comparable to the peptides in this study, although the hemolytic activity of this peptide was not tested (54). FKVKFKVKVK (SEQ ID NO:23) was shown by CD to adopt a β-sheet structure in the presence of either 50% TFE or 25 mM sodium dodecyl sulfate.

Recently, a series of (KL)ₙK—NH₂ (for n=7, 15 residues: SEQ ID NO:24) peptides containing 9 to 15 residues (all dansylated at the amino terminus) was studied by Castano et al. (55). The amide I vibrational band in the IR spectra (either dry, at the air/water interface, or inserted into a lipid monolayer) of these peptides was very similar to that of KIGAKIx3 (FIG. 3), centered near 1620 cm⁻¹. All of the peptides induced both leakage in lipid vesicles and hemolysis, with activity increasing as a function of length. The antimicrobial activity of these peptides was not examined.

Shai and coworkers studied diastereomeric antimicrobial peptides based upon 12-mers containing K and L (56, 57) or derivatives of pardaxin (58). These peptides were derived from all-L-amino-acid parent compounds that can form amphipathic α-helices. Based upon changes in the amide I infrared band, the conformation of the diastereomeric peptides was interpreted to be mainly β-sheet; however, the appearance of the amide I band is much different than that of the peptides in this study or those examined by Castano et al. (55). Instead of a relatively narrow band near 1620 cm⁻¹, the diastereomeric peptides gave rise to broad bands centered between 1640 and 1650 cm⁻¹. Clearly, the conformation of these peptides is significantly different than that of KIGAKI when bound to lipid bilayers.

### SUMMARY OF THE INVENTION

The present invention is directed to peptides which carry a sufficient positive charge to selectively disrupt microbial but not mammmalian cell membranes, contain enough hydrophobic residues to be able to enter a cell membrane, preferentially assume a beta sheet structure in a membrane environment, are substantially amphipathic in that structure but not in an alpha helical structure, and have antimicrobial activity.

All known naturally occurring linear cationic peptides adopt an amphipathic α-helical conformation upon binding to lipids as an initial step in the induction of cell leakage. We designed an 18-residue peptide, (KIGAKI)₃-NH₂ (SEQ ID NO:8), that has no amphipathic character as an α-helix but can form a highly amphipathic β-sheet. When bound to lipids, (KIGAKI)₃-NH₂ (SEQ ID NO:8) did indeed form β-sheet structure, as evidenced by Fourier transform infrared and circular dichroism spectroscopy. The antimicrobial activity of this peptide was comparable to that of (KIAGKIA)₃-NH₂ (SEQ ID NO:4), and better than that of PGLa (SEQ ID NO:2) and (KLAGLAK)₃-NH₂ (SEQ ID NO:6), all of which form amphipathic α-helices when bound to membranes. (KIGAKI)₃-NH₂ (SEQ ID NO:8) was much less effective at inducing leakage in lipid vesicles composed of mixtures of the acidic lipid, phosphatidylglycerol and the neutral lipid, phosphatidylcholine, as compared to the other peptides. When phosphatidylethanolamine replaced phosphatidylcholine, however, the lytic potency of PGLa and the α-helical model peptides was reduced, while that of (KIGAKI)₃-NH₂ (SEQ ID NO:8) was improved. Moreover, fluorescence experiments using analogs containing a single tryptophan residue showed that unlike the α-helical peptides, (KIGAKI)₃-NH₂ (SEQ ID NO:8) preferentially bound to vesicles containing phosphatidylethano-lamine instead of phosphatidylcholine, suggesting enhanced selectivity between bacterial and mammalian lipids.

Linear amphipathic β-sheet peptides such as (KIGAKI)₃-NH₂ (SEQ ID NO:8) may be used as antimicrobial agents.

### BRIEF DESCRIPTION OF THE DRAWINGS

FIG. 1. Helical wheel and beta strand diagrams showing the distribution of amino acid side chains (+=lysine, white=glycine, gray=alanine, and black=isoleucine or leucine) The hydrophobic moment (μ_{H}), calculated using the consensus hydrophobicity scale (15), is noted for each conformation. (A) (KIAGKIA)₃ (SEQ ID NO:4); (B) (KLAGLAK)₃ (SEQ ID NO:6); and (C) (KIGAKI)₃ (SEQ ID NO:8).

FIG. 2. CD spectra of (KIAGKIA)₃ (dashed line), (KLAGLAK)₃ (dash-dotted line), and (KIGAKI)₃ (solid line) in (A) aqueous buffer; (B) 50% trifluoroethanol/buffer; and (C) POPG LUV (lipid-to-peptide ratio=20).

FIG. 3. FTIR spectra of (KIAGKIA)₃ (A), (KLAGLAK)₃ (B), and (KIGAKI)₃ (C) in the presence of POPG (lipid-to-peptide ratio=20).

FIG. 4. Percent release of calcein from LUV three minutes following the addition of PGLa (open bars), (KIAGKIA)₃ (fine hatched bars), (KLAGLAK)₃ (coarse hatched bars), or (KIGAKI)₃ (cross-hatched). LUV composition: (A) POPG; (B) POPC; and (C) E. coli polar lipids (67% PE, 23% PG, 10% DPG)

FIG. 5. Percent release of calcein from LUV three minutes following the addition of PGLa (open bars), (KIAGKIA)₃ (fine hatched bars), (KLAGLAK)₃ (coarse hatched bars), or (KIGAKI)₃ (cross-hatched bars). LUV composition: (A) 1:1 POPC/POPG; (B) 2:1 POPC/POPG; (C) 3-1 POPC/POPG; (D) 4:1 POPC/POPG; (E) 1:1 POPE/POPG; (F) 2:1 POPE/POPG; (G) 3:1 POPE/POPG; and (H) 4:1 POPE/POPG.

FIG. 6. Percent release of calcein from LUV three minutes following the addition of PGLa (open bars), (KIAGKIA)₃ (fine hatched bars), (KLAGLAK)₃ (coarse hatched bars), or (KIGAKI)₃ (cross-hatched bars). LUV composition: (A) POPC/POPG/DPG (6.7/2.3/1); and (B) POPE/POPG/DPG (6.7/2.3/1). The replacement of PE by PC in the ternary mixture results in an increase in potency for all peptides except (KIGAKI)₃-NH₂.

FIG. 7. Shifts in the emission maximum of tryptophan fluorescence of W-KIAGKIA (SEQ ID NO:5) (A), W-KLAGLAK (SEQ ID NO:7) (B), and W-KIGAKI (SEQ ID NO:9) (C). The emission peak positions in aqueous solution were: 356 nm (W-KIAGKIA); 354 nm (W-KLAGLAK); and 355 nm (W-KIGAKI). The peptide concentration was 3 μM. The lipid-to-peptide ratio was 20 for measurements in the presence of LUV. The LUV abbreviations are: PC=POPC; PC/PG=POPC/POPG; PE/PG=POPE/POPG; PG=POPG; E. coli=E. coli polar lipids; PE/PG/DPG=POPC/POPG/DPG (6.7/2.3/1); and PC/PG/DPG=POPC/POPG/DPG (6.7/2.3/1). Errors are less than ±2 nm for all measurements.

We measured the shift for three peptides (W-KIAGKIA, W-KLAGLAK, and W-KIGAKI) to estimate binding to LUVs with different lipid composition at a lipid-to-peptide ratio of 20. For each peptide, the greatest shift occurred with POPG LUVs. The Trp analogue of (KIAGKIA)₃-NH₂ had a slightly higher affinity for LUVs containing POPC vs. POPE. The Trp analogue of (KLAGLAK)₃-NH₂ had a much higher affinity for LUVs containing POPC vs. POPE. In contrast, the Trp analogue of (KIGAKI)₃-NH₂ had a higher affinity for LUVs containing POPE vs. POPC.

FIG. 8: Model of Peptide Association and Disruption of the Lipid Bilayer.

PE promotes negative curvature in the bilayer surface because its head group is smaller than that of PC. If peptide induced disruption is the result of an increase in positive curvature, than the replacement of PC with PE should reduce leakage.

FIG. 9: Estimate of Peptide Molecules per Bacterium and Surface Lipid in MIC Assays

FIG. 10: Hydrophobic Moments of 18-Mer peptides (A) Alpha-Helical Conformation, (B) Beta-Sheet Conformation. Peptides: “1,2-Hex” (KKIGAI)₃ (SEQ ID NO:10); “1,3-Hex” (KIKGAI)₃ (SEQ ID NO:11); “1,4-Hex” (KIGKAI)₃ (SEQ ID NO:12); “1,5-Hex” (KIGAKI)₃ (SEQ ID NO:8); “1,6-Hex” (KIGAIK)₃ (SEQ ID NO:13); “Short-Hept” (KLAGKLA)₂KLAG (SEQ ID NO:14)

FIG. 11: Hydrophobic Moments of 21-Mer peptides (A) Alpha-Helical Conformation, (B) Beta-Sheet Conformation. Peptides: “1,2-Hept” (KKLAGLA)₃ (SEQ ID NO:15); “1,3-Hept” (KLKAGLA)₃ (SEQ ID NO:16); “1,4-Hept” (KLAKGLA)₃ (SEQ ID NO:17); “1,5-Hept” (KLAGKLA)₃ (SEQ ID NO:18); “1,6-Hept” (KLAGLKA)₃ (SEQ ID NO:19); “1,7-Hept” (KLAGLAK)3 (SEQ ID NO:6); “Long-Hex” (KIGAKI)₃ KIG (SEQ ID NO:20).

### DETAILED DESCRIPTION OF THE PREFERRED EMBODIMENTS OF THE INVENTION

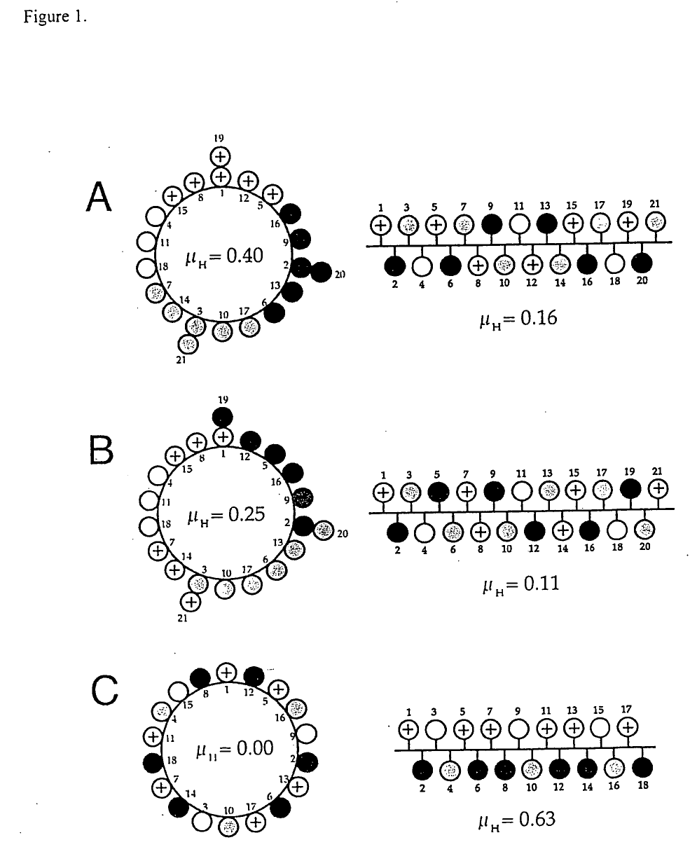

PGLa possesses greater Gram positive antimicrobial activity than magainin 2 (see Table 2). PGLa is largely α-helical when bound to lipid bilayers and appears to form pores in membranes. A more potent derivative of PGLa contains three heptamer repeats of sequence KXXXKXX, where X represents a nonpolar residue, as shown in Table 1. Cp. AIAGKIA in PGLa at residue 8-14 of SEQ ID NO:2. A 21-residue amidated peptide containing three heptameric repeats of KIAGKIA possesses high antimicrobial and relatively low hemolytic activity. When KIAGKIA¹ adopts an α-helical conformation, the peptide is highly amphipathic with all six lysines clustered on the helical face (FIG. 1). Using a is consensus hydrophobicity scale, the hydrophobic moment, μ_{H}, a quantitative measure of amphipathicity , for this peptide is much greater as an α-helix (0.40) as compared to a β-sheet (0.16).

In order to determine whether a highly amphipathic α-helix is a prerequisite for potent antimicrobial activity, we synthesized a peptide, KLAGLAK, with a similar amino acid content but with a heptamer repeat that separates the six lysines into two groups of three on the helical face, resulting in a large decrease in pH to 0.25 (see Table 1 and FIG. 1). Like KIAGKIA, KLAGLAK cannot form a highly amphipathic β-sheet structure.

Even though all known naturally occurring linear antimicrobial peptides have the capability to form at least a reasonably amphipathic α-helical structure, we designed a new peptide that can form a highly amphipathic β-sheet rather than α-helix. This 18-residue peptide contains the hexameric repeat, KIGAKI, (Table I). The value of μ_{H} as an α-helix and a β-sheet is 0 and 0.63, respectively, as shown in FIG. 1. The three model peptides, KIAGKIA, KLAGLAK, and KIGAKI, possess equal charge (+7) and nearly equal mean hydrophobicity values. We compared the antimicrobial and hemolytic activity of these peptides, used CD and FTIR spectroscopy to determine the conformation of the peptides in solution and when bound to lipid bilayers, and measured the ability of the peptides to induce leakage in and bind to LUV of varying lipid composition. Our results show that KIGAKI does indeed adopt a β-sheet conformation when bound to lipids and is comparable in antimicrobial activity to KIAGKIA and KLAGLAK. KIGAKI appears to possess greater selectivity for bacterial vs. mammalian lipids as compared to the α-helical peptides tested.

The appended claims are hereby incorporated by reference as an enumeration of some of the preferred embodimens.

Abbreviations

CD circular dichroism

DSC differential scanning calorimetry

FTIR Fourier transform infrared

L/P lipid-to-peptide ratio

LUV large unilamellar vesicle

μ_{H} hydrophobic moment

MIC minimum inhibitory concentration

DiPoPE 1,2-dipalmitoleoylphosphatidylethanolamine

DPG diphosphatidylglycerol

PC phosphatidylcholine

PE phosphatidylethanolamine

PG phosphatidylglycerol

POPC 1-palmitoyl-2-oleoylphosphatidylcholine

POPE 1-palmitoyl-2-oleoylphosphatidylethanolamine

POPG 1-palmitoyl-2-oleoylphosphatidylglycerol

T_{H} bilayer-to-hexagonal phase transition temperature

TFE trifluoroethanol

LPS lipopolysaccharide

NMR nuclear magnetic resonance

REDOR rotational echo double resonance (form of NMR)

CFU colony forming units

EDTA ethylene diamine tetraacetic acid

PIPES piperazine-N,N′-bis(2-ethanesulfonic acid)

HEPES N-(2-hydroxyethyl)piperazine-N′(2-ethane sulfonic acid)

ONGP o-nitrophenyl-β-D-galactopyranoside

HPLC high pressure liquid chromatography

Cationicity

A “cationic” peptide is one having a net positive charge. Among the 20 genetically encoded amino acids, only Arg and Lys have a full positive charge (+1 each) under normal physiological conditions. His has a partial positive charge (its pK varies with the environment, but is usually about 6.8; at pH 7, the charge would be +0.4). In contrast, Asp and Glu have have full negative charge (−1 each).

With a normal peptide, one terminal is NH2—, with a charge of +1, and the other is —COOH, with a charge of −1, so the termini balance each other out. However, it is possible to amidate the second end of the peptide so that it is —NH2 instead of —COOH.

The peptides of the present invention comprise one or more positively charged amino acids, so that they have a net positive charge. The net positive charge must be sufficient for the peptide to have some antimicrobial activity.

Preferably, the peptides of the present invention are cationic peptides with a net charge of at least +4 (like magainin 2), more preferably at least +5 (like PGLa), still more preferably at least +6, most preferably at least +7.

If the charge is too high, selectivity is diminished. The net positive charge must not be so high that there is a complete loss of selectivity between microbial and mammalian cells. Preferably, the net positive charge is not more than +10.

Charge Density

The “charge density” is the net total charge of the peptide, divided by the length of the peptide in amino acids. It is preferable that the charge density as defined above be in the range of 0.25 to 0.5. A lower charge density than 0.25 would imply a longer molecule if the net charge were held constant, leading possibly to lower yields. A higher charge density than 0.5 would limit the amphipathicity of the peptide in the beta sheet state (see below), as it then would not be possible to alternate hydrophilic (all positively charged residues are hydrophilic) and hydrophobic residues.

Considerations of amphipathicity alone would point toward a most preferred charge density of 0.5. However, it is desirable to saturate the membrane's negative charges with as great a mass of peptides as possible. Use of a peptide that is too highly charged could result in a lower bound mass, through peptide/peptide repulsive effects, or “overbinding” (a peptide interacting with more membrane negative charges than is necessary for adequate binding, thereby denying use of the excess bound negative charges to another peptide). In the successful (KIGAKI)3 peptide of the present invention, the charge density is 0.33.

For peptidomimetics, the charge density may be expressed as the net charge divided by the length of the molecule in angstroms. In a beta-sheet, the translation per residue is 3.2 (parallel) to 3.4 (antiparallel) angstroms. Hence, the charge density, when expressed in charge/angstroms, is 0.075 to 0.15.

Hydrophobicity/Hydrophilicity Scales

In order to calculate the total or mean hydrophobicity, or the amphipathicity, of a peptide, it is necessary to select a hydrophobicity scale. The hydrophobicity scale is an an attempt to quantify the preference of the amino acids for polar (esp. aqueous) and nonpolar (esp. lipid) environments. The quantitative and even qualititative differences between the scales is, perhaps, not surprising, given that it is unrealistic to expect that all aspects of the interaction of a residue with water, with lipid, and with other residues in the peptide or protein can be summarized in a single number.

In a general sense, an amino acid may be considered to have some hydrophobic characteristics if its value on any art-accepted scale is of a sign (usually positive) which is representative, on that scale, of hydrophobicity.

However, to quantify the desired hydrophobicity of a peptide, it is necessary to pick a particular scale.

Hence, unless otherwise stated, it should be assumed that any quantitative teaching concerning hydrophobicity is based on Eisenberg's consensus hydrophobicity scale.

This scale is a simple average of four other scales, those of von Heijne, Janin, Chothia, and Wolfenden. The von Heijne scale was theoretical, describing the energetic effects of the covering of hydrophobic surface area, hydrogen-bond breakage, and charge neutralization. Janin's scale is based on the fraction of each type of residue that is found buried in globular proteins. Chothia looked at the observed distribution of amino acid side chains between the surface and the interior of proteins. Finally, Wolfenden tabulated the Gibbs free energy of transfer from dilute aqueous solution to the vapor of substances of the class RH, where R represents an amino acid side chain (e.g., RH for glycine is H2).

Another experimental scale is that of Nozaki, which is based on the free energy of transfer of amino acids from water to ethanol, but which, unfortunately, as originally published, was incomplete. Segrest and Feldman have suggested values for some of the omitted residues.

Another consensus scale is that of Kyte and Doolittle, which considers both the values from water-to-vapor transfers and the internal-external distribution of amino acid residues, adjusted subjectively.

The Argos “membrane-buried preference” scale is derived from the relative frequencies of 1125 amino acids found in protein segments judged to be within membranes.

The OMH (optimal matching hydrophobicity) scale is derived from sequence alignments, and assumes that families of proteins that fold the same way do so because they have the same pattern of residue hydrophobicities along their amino acid sequences.

The Eisenberg consensus scale has been established only for the genetically encoded amino acids. For other amino acids, one must rely on a purely experimental scale, such as that of Wolfenden or Nozaki. Use of the Wolfenden scale is preferred because it is a component of the Eisenberg consensus scale.

If a peptide includes amino acids for which there is no established Eisenberg consensus scale value, the procedure is, for each such amino acid (1) determine its value experimentally according to an experimental scale, such as the Wolfenden scale, and (2) determine its equivalent value on the Eisenberg consensus scale by finding the least squares fit between the experimental scale value for the genetically encoded AAs and the Eisenberg consensus scale value for those AAs.

For convenience, the Eisenberg and Wolfenden scales are set forth below:

| Amino | Eisenberg | Wolfenden |
|---|---|---|
| Acid | Hydrophobicity | Hydrophobicity |
| Ile | 0.73 | 2.15 |
| Phe | 0.61 | −0.76 |
| Val | 0.54 | 1.99 |
| Leu | 0.53 | 2.28 |
| Trp | 0.37 | −5.88 |
| Met | 0.26 | −1.48 |
| Ala | 0.25 | 1.94 |
| Gly | 0.16 | 2.39 |
| Cys | 0.04 | −1.24 |
| Tyr | 0.02 | −6.11 |
| Pro | −0.07 | — |
| Thr | −0.18 | −4.88 |
| Ser | −0.26 | −5.06 |
| His | −0.40 | −10.27 |
| Glu | −0.62 | −10.20 |
| Asn | −0.64 | −9.68 |
| Gln | −0.69 | −9.38 |
| Asp | −0.72 | −10.95 |
| Lys | −1.1 | −9.52 |
| Arg | −1.8 | −19.92 |

Mean Hydrophobicity

Cationic peptides are typical soluble in water. However, to interact with the bacterial membrane, solubility in water is not sufficient. The bacterial membrane is amphipathic in nature, with the hydrophilic moieties on the outside and the hydrophobic moieties on the inside. For a cationic peptide to interact with the hydrophobic moieties of the membrane, it must have a hydrophobic component. However, the positively charged amino acids are highly hydrophilic. Hence, the peptides of the present invention will also include one or more hydrophobic moieties.

Preferably, the arithmetic mean hydrophobicity of the peptide is preferably at least −0.8, more preferably at least −0.6, with the-individual hydrophobicities being determined according to Eisenberg's non-normalized consensus hydrophobicity scale. An (RI)n oligomer will have a mean hydrophobicity of −0.535 ((−1.8+0.73)/2), a (KI)n oligomer of −0.185, an (HI)n oligomer of +0.165, an RL oligomer of −0.435, a (KL)n oligomer of −0.285, etc.

By way of comparison, the mean hydrophobicities are −0.0357 for magainin 2; −0.1832 for MSI-78; +0.0381 for PGLa.

Peptide Length

In order to achieve both the charge density desiderata, a peptide length of 2*p to 4*p, where p is the number of positively charged amino acids, is desirable (as it yields the preferred charge density of 0.5 to 0.25). Since the net positive charge is preferably at least +4, the preferred minimum length is at least 8 a.a.'s.

The preferred maximum peptide length is set by considerations of yield and cost. Preferably, the peptides are not more than 50 AA, more preferably not more than 30 AA, still more preferably not more than 20 AA.

The peptides may be composed of 2, 3 or more perfect or nearly perfect repeats of a 6-8 amino acid repeat sequence, such as KIGAKI (SEQ ID NO:8, residues 1-6).

Secondary Structure

A peptide may assume a variety of secondary structures, the most common of which are the (right-handed) alpha-helix and beta-sheet (strand). While the term beta-strand is probably more accurate for the short peptides contemplated here than is beta-sheet, the literature refers much more often to beta-sheets than to beta-strands, and so that convention is followed here.

An alpha helix is stabilized by intramolecular hydrogen bonding between the amino and carboxyl groups of the peptide backbone. The structure resembles a coil with 3.6 amino acid residues per turn, and a translation of 1.5 angstroms per residue. Each amino acid side chain, extending out from the coiled backbone, is offset by 100 degrees from its nearest neighbors when viewed down the long helical axis.

A beta sheet (strand) consists of an extended peptide chain that can be stabilized by either intramolecular (strand or sheet) or intermolecular (sheet only) hydrogen bonds. In a beta strand, each amino acid side chain extends in the opposite direction (180 deg. offset) from its nearest neighbors.

The peptides of the present invention are those which substantially prefer the beta-sheet structure over the alpha-helix structure when bound to a lipid bilayer or a microbial membrane.

More particularly, as measured by circular dichroism and infrared spectroscopy, when bound to a lipid vesicle model of a bacterial membrane (the peptide secondary structure can not be determined when the peptides are bound to a bacterial membrane, since the latter is already loaded with protein), they are preferably more than 50%, still more preferably at least 80%, even more preferably at least 90%, most preferably at least 95%, in the beta sheet conformation, when in a bacterial membrane or in a lipid vesicle model of a bacterial membrane.

Amphipathicity

An amphipathic peptide is one which, in its principal 3D secondary structure conformation, has a hydrophobic face and a hydrophilic face. (The terms amphipathic and amphiphilic are interchangeable.)

A quantitative value for the degree of amphipathicity can be assigned using Eisenberg's consensus hydrophobicity scale and his definition for mean hydrophobic moment (μ_{H}).¹⁴ The hydrophobic moment is defined as

$${\{ {[ {\sum }_{n = 1}^{N} {H}_{n} sin ( δ n ) ]}^{2} + {[ {\sum }_{n = 1}^{N} {H}_{n} cos ( δ n ) ]}^{2} \}}^{1 / 2}$$

for an amino acid sequence of N residues, where the individual residue hydrophobicities are Hn for residue n, and δ is the angle (in radians) at which successive side chains emerge from the backbone when the periodic segment is viewed down its axis. This angle is 2Π*(100/360) radians for an alpha helix, and Π radians for a beta-sheet.

It can be seen that the hydrophobic moment is the vector sum of the individual hydrophobicities. The value of the moment is sensitive to the hydrophobicity scale employed, and hence the preferred moments set forth below are to be understood as calculated using the Eisenberg scale.

According to the Eisenberg scale, for the genetically encoded amino acids, arginine (−1.8) is the most hydrophilic amino acid, and isoleucine (0.73) is the most hydrophobic amino acid. Thus, the most amphipathic peptide constructible from the genetically encoded amino acids will be composed just of arginine and isoleucine.

We will use the symbol μ_{H}α for the hydrophobic moment of the peptide in its alpha-helical conformation (the “alpha moment”), and μ_{H}β for its moment in its beta-sheet conformation (the “beta moment”).

By way of comparison, for magainin 2, μ_{H} has a value of 0.286 as an alpha-helix, and 0.037 as a beta-sheet. For MSI-78, the moments are 0.449 (alpha) and 0.143 (beta). For PGLa, the moments are 0.264 (alpha) and 0.07 (beta).

The peptides of the present invention are preferably substantially more amphipathic as beta strands than as alpha helices.

Preferably, the alpha moment of the peptides of the present invention is less than 0.2, more preferably less than 0.1, still more preferably less than 0.05.

Preferably, the beta moment of the peptides of the present invention is greater than 0.2, more preferably greater than 0.4.

Preferably, the beta moment for the peptides of the present invention is at least 0.2 higher than the alpha moment.

The difference between the moments is probably more significant than the absolute values, as it is the difference which is primarily responsible for the stabilization of the peptide in a membrane or membranomimetic system into a beta-sheet secondary structure. For example, in water, or in a TFE/water mixed solvent, the (KIGAKI)3 peptide is mainly alpha-helical in character. It is only in a lipid bilayer, where the amphipathic beta-sheet is stabilized relative to the non-amphipathic alpha-helix, that the beta-sheet structure predominates. The amphipathic beta-sheet aligns with the bilayer so that the hydrophobic face is on the lipid side of the bilayer and the hydrophilic face is on the aqueous side of the bilayer.

Composition

The peptide of the present invention will necessarily comprise one or more positively charged amino acids, such as lysine, arginine, and histidine. Lysine and Arginine are preferred to histidine because they have a full positive charge. These positively charged amino acids will normally be in positions which place them in the hydrophilic face of the desired beta sheet structure. The hydrophilic face may also include uncharged/hydrophilic AAs like Ser, neutral AAs like Gly (which is in our preferred peptide (KIGAKI)3). It less desirable for it to include negatively charged AAs (which lower the net charge), or hydrophobic Aas (of the latter, those which can form H-bonds are preferred).

The peptides of the present invention also will comprise one or more hydrophobic amino acids, such as Leu, Ile, Val, Met, Phe, Trp, Tyr, or Ala. In general, for amino acids which are to be part of the hydrophobic face of the desired beta-sheet structure, the more hydrophobic the AA, the better.

Certain amino acids present synthetic difficulties; these are isoleucine (because of the location of the beta branch); methionine (because it can oxidize); and Cys (because of its propensity to form disulfide bonds, which can cause the peptides to form multimolecular aggregates. It is noted that the defensins are natural antimicrobial peptides of about 30 a.a. with an intrinsic beta sheet structure becasue of three disulfide bonds. However, these disulfide bonds make it difficult to synthesise the defensins because of the difficulty of controlling disulfide bond topology.) However, none of these AAs are absolutely prohibited and Ile, despite its adverse effect on synthetic yield, remains a preferred amino acid for the hydrophobic face because of its very high hydrophobicity.

Scientists have determined the propensity of various amino acids to appear in alpha helices or beta sheets of proteins. This propensity data is of only marginal value in the present context, where we are looking at the structure of small peptides in a lipid bilayer environment. There are a variety of interactions at work in the interior of a protein which are not applicable in our context. Our peptides are too small to have a stable secondary structure in solution, so it is expected that the relative amphipathicity of the peptide in the beta-sheet as opposed to the alpha-helix state is what will primarily drive which structure it assumes in the lipid bilayer.

Nonetheless, in comparing two peptides with similar relative amphipathicities, these secondary structure propensities, and especially the relative propensities, of the individual amino acids may be relevant.

| | (1) Chou- | (2) Chou- | Relative |
|---|---|---|---|
| | Fasman (1974) | Fasman (1974) | Propensity |
| Amino | Alpha-Helix | Beta-Sheet | (ratio of (1) |
| Acid | Propensity | Propensity | to (2)) |
| Ala | 1.45 | 0.97 | 1.494845361 |
| Cys | 0.77 | 1.30 | 0.592307692 |
| Asp | 0.98 | 0.80 | 1.225 |
| Glu | 1.53 | 0.26 | 5.884615385 |
| Phe | 1.12 | 1.28 | 0.875 |
| Gly | 0.53 | 0.81 | 0.654320988 |
| His | 1.24 | 0.71 | 1.746478873 |
| Ile | 1.00 | 1.60 | 0.625 |
| Lys | 1.07 | 0.74 | 1.445945946 |
| Leu | 1.34 | 1.22 | 1.098360656 |
| Met | 1.2 | 1.67 | 0.718562874 |
| Asn | 0.73 | 0.65 | 1.123076923 |
| Pro | 0.59 | 0.62 | 0.951612903 |
| Gln | 1.17 | 1.23 | 0.951219512 |
| Arg | 0.79 | 0.9 | 0.877777778 |
| Ser | 0.79 | 0.72 | 1.097222222 |
| Thr | 0.82 | 1.2 | 0.683333333 |
| Val | 1.14 | 1.65 | 0.690909091 |
| Trp | 1.14 | 1.19 | 0.957983193 |
| Tyr | 0.61 | 1.29 | 0.472868217 |

Similarly, one may take into account the reported tendencies of homopolymers of oligopeptides to form alpha-helices or beta-sheets in water. However, this is with the caveat that we are concerned with the formation of secondary structure in a lipid bilayer, rather than in water. Below are reported helix formation parameters for amino acid residues; these give the equilibrium constants for initial helical conformation in a random coil or for adding a residue of helical conformation to the end of a helix. The table below is for 18 of the amino acids:

| | helix | |
|---|---|---|
| Amino | initiation | helix |
| Acid | (×10E4) | addition |
| Gly | 0.1 | 0.6 |
| Asp− | 70 | 0.66 |
| His+ | 0.1 | 0.68 |
| Ser | 0.1 | 0.77 |
| Asn | 0.1 | 0.79 |
| His | 210 | 0.80 |
| Thr | 0.1 | 0.83 |
| Lys+ | 1 | 0.94 |
| Val | 1 | 0.96 |
| Gln | 33 | 0.96 |
| Glu− | 6 | 0.96 |
| Tyr | 66 | 0.99 |
| Arg+ | 0.1 | 1.02 |
| Ala | 8 | 1.06 |
| Trp | 77 | 1.08 |
| Phe | 18 | 1.08 |
| Ile | 55 | 1.12 |
| Leu | 33 | 1.14 |
| Met | 54 | 1.17 |

Sequence

The mean hydrophobicity is determined completely by the AA composition of the peptide. However, the amphipathicity is a function of both composition and sequence.

For a beta-sheet, a perfectly amphipathic sequence has the structure (wl)ₙ or (lw)ₙ, where w denotes a hydrophilic (water-loving) amino acid, 1 a hydrophobic (lipid-loving) amino acid, and n is a positive integer. In other words, the maximum amphipathicity is reached when the hydrophobic and hydrophilic amino acids alternate.

If all of the hydrophilic amino acids are positively charged, this perfectly amphipathic sequence would have a charge density of 0.5. If only half were positively charged, and the rest uncharged, this perfectly amphipathic sequence would have a charge density of 0.25.

The peptides of the present invention are preferably perfectly amphipathic as beta-strands. If they are not perfectly amphipathic, they are preferably nearly perfectly amphipathic, i.e., the only departure from perfect amphipathicity is the presence of one or more Gly (a neutral AA) in one or both faces.

However, it is possible for the peptide to depart further from perfect amphipathicity, i.e., have a hydrophobic AR in the hydrophilic face, or vice versa, although it preferably still satisfies the aforementioned preference concerning the values of the alpha and beta moments.

For an alpha helix, a perfectly amphipathic 18 a.a. sequence would be (wwllwwllw llwwllwwl)ₙ, or (llwwllwwl wwllwwllw)ₙ.

If we line up the sequences for a perfectly amphipathic 18 a.a. beta and a perfectly amphipathic 18 a.a. alpha, we have

| | |
|---|---|
| wlwlwlwlwlwlwlwlwl | (beta) |
| wwllwwllwllwwllwwl | (alpha) |

and three permutations thereof.

For an 18 a.a. peptide composed of nine Arg and nine Ile, the maximum beta moment would be achieved if the residues were arranged in the sequence (RI)₉, and would be 1.27 (This sequence would have an alpha moment of 0.0). The maximum alpha moment would be for the sequence RRIIRRIIR IIRRIIRRI, and would be 0.81 (This sequence would have a beta moment of 0.14).

Increasing or decreasing the length of a peptide which was 50% R 50% I would not alter the maximum achievable beta moment, which would be with (RI)n or (IR)n. It would alter the maximum achievable alpha moment, for example, for 8R/8I, it would be 0.85, and for 10R/10I, it would be 0.83.

Predicted Helical Character

Agadir is a prediction algorithm based on the helix/coil transition theory. Agadir predicts the helical behaviour of monomeric peptides. It only considers short range interactions.

Unfortunately, the Agadir algorithm is based on the behavior of peptides in aqueous solution. It does not consider the role of amphipathicity in stabilizing one structure over another. Also, it only considers the helix/coil, and not the beta-strand/coil transition.

Still, for peptides of equal amphipathicity, it may be of interest to look at the relative helical character predicted according to the Agadir algorithm. For (RI)9, the predicted percentage helix was 1.97%; for (KIGAKI)3, it was 0.85%; for RRIIRRIIRIIRRIIRRI, it was 5.13%; for magainin 2, 0.83%; MSI-78, 2.18%; PGLa, 3.29%; all for N-terminal free, C-terminal amidated peptides under the default conditions of pH 7.00, Temperature 278, Ionic Strength 0.100. For melittin, with free N and C termini, it was 2.04%, and, C-amidated, 2.11%.

Preferably, the predicted percentage helical character of the peptide in aqueous solution, according to the Agadir algorithm, under its default conditions, for the peptide in amidated form, is less than 2%.

The Agadir algorithm is described in Muñoz, V. & Serrano, L. (1994a)., Elucidating the folding problem of helical peptides using empirical parameters. Nature: Struct. Biol. 1, 399-409; Muñoz, V. & Serrano, L. (1994b). Elucidating the folding problem of α-helical peptides using empirical parameters, II. Helix macrodipole effects and rational modification of the helical content of natural peptides. J. Mol. Biol 245, 275-296; Muñoz, V. & Serrano, L. (1994c). Elucidating the folding problem of α-helical peptides using empirical parameters III:Temperature and pH dependence J. Mol. Biol 245, 297-308; Mufioz, V. & Serrano, L. (1997). Development of the Multiple Sequence Approximation within the Agadir Model of a-Helix Formation. Comparison with Zimm-Bragg and Lifson-Roig Formalisms. Biopolymers 41, 495-509; Lacroix, E., Viguera AR & Serrano, L. (1998) Elucidating the folding problem of α-helices: Local motifs, long-range electrostatics, ionic strength dependence and prediction of NMR parameters. J. Mol. Biol. 284, 173-191; Villegas, V., Viguera, A. R., AvilΘs, F. X. & Serrano, L. (1996). Stabilisation of proteins by rational design of α-helix stability using helix/coil transition theory. Folding & Design, 1,29-34; Muñoz, V. & Serrano, L. (1996). Local vs non-local interactions in protein folding and stability. An experimentalist point of view. Folding & Design. 1, R71-R77; ≦pez-Hernβndez, E., Cronet, P., Serrano, L. & Muñoz, V. (1997). Kinetics of CheY mutants with enhanced native α-helix propensities. J. Mol. Biol. 266, 610-620. Viguera, A. R., Villegas, V., AvilΘs, F. X. & Serranor L. (1996). Favourable native-like helical local interactions can accelerate protein folding. Folding & Design, 2, 23-33.

The EMBL WWW Gateway to AGADIR is at http://www.embl-heidelberg.de/Services/serrano/agadir/agadir-start.html

Should this URL change, the new URL may be identified by contacting or exploring the root domain, or, if that fails, by an internet search on the term “agadir”.

Amino Acids and Peptides

Amino acids are the basic building blocks with which peptides and proteins are constructed. Amino acids possess both an amino group (—NH₂) and a carboxylic acid group (—COOH). Many amino acids, but not all, have the structure NH₂—CHR—COOH, where R is hydrogen, or any of a variety of functional groups.

Twenty amino acids are genetically encoded: Alanine (A), Arginine (R), Asparagine (N), Aspartic Acid (D), Cysteine (C), Glutamic Acid (E), Glutamine (Q), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), and Valine (V) Of these, all save Glycine are optically isomeric, however, only the L-form is found in humans. Nevertheless, the D-forms of these amino acids do have biological significance; D-Phe, for example, is a known analgesic.

Many other amino acids are also known, including: 2-Aminoadipic acid; 3-Aminoadipic acid; beta-Aminopropionic acid; 2-Aminobutyric acid; 4-Aminobutyric acid (Piperidinic acid);

6-Aminocaproic acid; 2-Aminoheptanoic acid; 2-Aminoisobutyric acid, 3-Aminoisobutyric acid; 2-Aminopimelic acid;

2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline;

4-Hydroxyproline; Isodesmosine; allo-Isoleucine; N-Methylglycine (Sarcosine); N-Methylisoleucine; N-Methylvaline; Norvaline; Norleucine; and Ornithine.

Peptides are constructed by condensation of amino acids and/or smaller peptides. The amino group of one amino acid (or peptide) reacts with the carboxylic acid group of a second amino acid (or peptide) to form a peptide (—NHCO—) bond, releasing one molecule of water. Therefore, when an amino acid is incorporated into a peptide, it should, technically speaking, be referred to as an amino acid residue.

A peptide is composed of a plurality of amino acid residues joined together by peptidyl (—NHCO—) bonds. A biogenic peptide is a peptide in which the residues are all genetically encoded amino acid residues; it is not necessary that the biogenic peptide actually be produced by gene expression.

The peptides of the present invention include peptides whose sequences are disclosed in this specification, or sequences differing from the above solely by no more than one nonconservative substitution and/or one or more conservative substitutions, preferably no more than a single conservative substitution. The substitutions may be of non-genetically encoded (exotic) amino acids, in which case the resulting peptide is nonbiogenic.

A conservative substitution is a substitution of one amino acid for another of the same exchange group, the exchange groups being defined as follows

I Gly, Pro, Ser, Ala (Cys) (and any nonbiogenic, neutral amino acid with a hydrophobicity not exceeding that of the aforementioned a.a.'s)

II Arg, Lys, His (and any nonbiogenic, positively-charged amino acids)

III Asp, Glu, Asn, Gln (and any nonbiogenic negatively-charged amino acids)

IV Leu, Ile, Met, Val (Cys) (and any nonbiogenic, aliphatic, neutral amino acid with a hydrophobicity too high for I above)

V Phe, Trp, Tyr (and any nonbiogenic, aromatic neutral amino acid with a hydrophobicity too high for I above).

Note that Cys belongs to both I and IV.

A highly conservative substitution, which is preferred, is Arg/Lys/His, Asp/Glu, Asn/Gln, Leu/Ile/Met/Val, Phe/Trp/Tyr, or Gly/Ser/Ala.

Additional peptides witin the present invention may be identified by systematic mutagenesis of the lead peptides, e.g.

(a) separate synthesis of all possible single substitution (especially of genetically encoded AAs) mutants of each lead peptide, and/or

(b) simultaneous binomial random alanine-scanning mutagenesis of each lead peptide, so each amino acids position may be either the original amino acid or alanine (alanine being a semi-conservative substitution for all other amino acids), and/or

(c) simultaneous random mutagenesis sampling conservative substitutions of some or all positions of each lead peptide, the number of sequences in total sequences space for a given experiment being such that any sequence, if active, is within detection limits (typically, this means not more than about 10¹⁰ different sequences).

The mutants are tested-for activity, and, if active, are considered to be within “peptides of the present invention”. Even inactive mutants contribute to our knowledge of structure-activity relationships and thus assist in the design of peptides, peptoids, and peptidomimetics.

Preferably, substitutions of exotic amino acids for the original amino acids take the form of

(I) replacement of one or more hyd-rophilic amino acid side chains with another hydrophilic organic radical, not more than twice the volume of the original side chain, or

(II) replacement of one or more hydrophobic amino acid side chains with another hydrophobic organic radical, not more than twice the volume of the original side chain.

The exotic amino acids may be alpha or non-alpha amino acids (e.g., beta alanine). They may be alpha amino acids with 2 R groups on the Cα, which groups may be the same or different. They may be dehydro amino acids (HOOC—C(NH₂)═CHR).

Exotic amino acids of particular interest include those which differ from a genetically encoded amino acid primarily by including more or fewer carbons, e.g., 5 or 6 carbon analogues of Leu, Ile or Val, or analogues of Lys with more or less than 4 carbons.

For further information on synthesis of peptides including exotic amino acids, see:

1. Bielfeldt, T., Peters, S., Meldal, M., Bock, K. and Paulsen, N.A. new strategy for solid-phase synthesis of O-glycopeptides. Angew. Chem. (Engl) 31:857-859, 1992.

2. Gurjar, M. K. and Saha, U. K. Synthesis of the glycopeptide-O-(3,4-di-O-methyl-2-O-[3,4-di-O-methyl-α-L-rhamnopyranosyl]-α-L-rhamnophyranosyl)-L-alanilol: An unusual part structure in the glycopeptidolipid of Mycobacterium fortuitum. Tetrahedron 48:4039-4044, 1992.

3. Kessler, H., Wittmann, V., Kock, M. and Kottenhahn, M. Synthesis of C-glycopeptides via free radical addition of glycosyl bromides to dehydroalanine derivatives. Angew. Chem. (Engl.) 31:902-904, 1992.

4. Kraus, J. L. and Attardo, G. Synthesis and biological activities of new N-formylated methionyl peptides containing an α-substituted glycine residue. European Journal of Medicinal Chemistry 27:19-26, 1992.

5. Mhaskar, S. Y. Synthesis of H-lauroyl dipeptides and correlation of their structure with surfactant and antibacterial properties. J. Am. Oil Chem. Soc.69:647-652, 1992.

6. Moree, W. J., Van der Marel, G. A. and Liskamp, R. M. J. Synthesis of peptides containing the β-substituted aminoethane sulfinamide or sulfonamide transition-state isostere derived from amino acids. Tetrahedron Lett. 33:69-6392, 1992.

7. Paquet, A. Further studies on the use of 2,2,2-trichloroethyl groups for phosphate protection in phosphoserine peptide synthesis. International Journal of Peptide and Protein Research 39:82-86, 1992.

8. Sewald, N., Riede, J., Bissinger, P. and Burger, K. A new convenient synthesis of 2-trifluoromethyl substituted aspartic acid and its isopeptides. Part 11. Journal of the Chemical Society. Perkin Transactions 1 1992:267-274, 1992.

9. Simon, R. J., Kania, R. S., Zuckermann, R. N., Huebner, V. D., Jewell, D. A., Banville, S., Ng, S., Wang, L., Rosenberg, S., Marlowe, C. K., Spellmeyer, D. C., Tan, R., Frankel, A. D., Santi, D. V., Cohen, F. E. and Bartlett, P. A. Peptoids: A modular approach to drug discovery. Proc. Natl, Acad. Sci. USA 89:9367-9371, 1992.

10. Tung, C.-H., Zhu, T., Lackland, H. and Stein, S. An acridine amino acid derivative for use in Fmoc peptide synthesis. Peptide Research 5:115-118, 1992.

11. Elofsson, M. Building blocks for glycopeptide synthesis: Glycosylation of 3-mercaptopropionic acid and Fmoc amino acids with unprotected carboxyl groups. Tetrahedron Lett. 32:7613-7616, 1991.

12. McMurray, J. S. Solid phase synthesis of a cyclic peptide using Fmoc chemistry. Tetrahedron Letters 32:7679-7682, 1991.

13. Nunami, K.-I., Yamazaki, T. and Goodman, M. Cyclic retro-inverso dipeptides with two aromatic side chains. I. Synthesis. Biopolymers 31:1503-1512, 1991.

14. Rovero, P, Synthesis of cyclic peptides on solid support. Tetrahedron Letters 32:2639-2642, 1991.

15. Elofsson, M., Walse, B. and Kihlberg, J. Building blocks for glycopeptide synthesis: Glycosylation of 3-mercaptopropionic acid and Fmoc amino acids with unprotected carboxyl groups. Tetrahedron Letter, 32:7613-7616, 1991.

16. Bielfeldt, T., Peter, S., Meldal, M., Bock, K. and Paulsen, H. A new strategy for solid-phase synthesis of O-glycopeptides. Agnew. Chem (Engl) 31:857-859, 1992.

17. Luning, B., Norberg, T. and Tejbrant, J. Synthesis of glycosylated amino acids for use in solid phase glycopeptide synthesis, par 2:N-(9-fluorenylmethyloxycarbonyl)-3-O-[2,4,6-tri-O-acetyl-α-D-sylopyranosyl)-β-D-glucopyranosyl]-L-serine. J. Carbohydr. Chem. 11:933-943, 1992.

18. Peters, S., Bielfeldt, T., Meldal, M., Bock, K. and Paulsen, H. Solid phase peptide synthesis of mucin glycopeptides. Tetrahedron Lett. 33:6445-6448, 1992.

19. Urge, L., Otvos, L., Jr., Lang, E., Wroblewski, K., Laczko,I. and Hollosi, M. Fmoc-protected, glycosylated asparagines potentially useful as reagents in the solid-phase synthesis of N-glycopeptides, Carbohydr. Res. 235:83-93, 1992.

20. Gerz, M., Matter, H. and Kessler, H., S-glycosylated cyclic peptides, Angew. Chem. (Engl.) 32:269-271, 1993.

N— and C-terminal Modified Peptides

In an unmodified peptide, one end of the peptide terminates in a —NH₂ and the other end has a free —COOH. If the —COOH is replaced with an —NH₂, the peptide is called an amide. The present invention includes the amide of any disclosed standard peptide.

In addition, further N— and C-terminal modifications are contemplated. The NH₂— and/or —COOH termini may be replaced with a group of the form RY—, where R is a hydrophobic moiety, and Y is a spacer. The resulting modified peptide should still have a solubility in water of at least 1 g/L at 20° C. Y may, for example, be —O—, —C(═O)—, or —C(═O)—NH—. R may be a hydrocarbon. If a hydrocarbon, R may be aliphatic or aromatic, and linear, branched or cyclic, and may contain alkenyl or alkynyl moieties. It is preferably alkyi of 2 to 12 carbons.

Modifications of particular interest include N-terminal alkanoyl modification (linear or branched chains, from 4- to 12 carbons), cyclic modification (e.g., cyclohexanoyl, etc.), or aromatic modification (e.g., benzyol, etc.).

If both N— and C-termini are modified, the modifications may be the same or different. It is noted that cyclic peptides constitute a special case of N— and C-terminal modification.

The term “peptide” is understood to include both modified and unmodified peptides, if not further qualified.

Cyclic Peptides

Many naturally occurring peptide are cyclic. Cyclization is a common mechanism for stabilization of peptide conformation thereby achieving improved association of the peptide with its ligand and hence improved biological activity. Cyclization is usually achieved by intra-chain cystine formation, by formation of peptide bond between side chains or between N— and C-terminals. Cyclization was usually achieved by peptides in solution, but several publications have appeared recently that describe cyclization of peptides on beads (see references below).

1. Spatola, A. F., Anwer, M. K. and Rao, M. N. Phase transfer catalysis in solid phase peptide synthesis. Preparation of cycle [Xxx-Pro-Gly-Yyy-Pro-Gly] model peptides and their conformational analysis. Int. J. Pept. Protein Res. 40:322-332, 1992.

2. Tromelin, A., Fulachier, M.-H., Mourier, G. and Menez, A. Solid phase synthesis of a cyclic peptide derived from a curaremimetic toxin. Tetrahedron Lett. 33:5197-5200, 1992.

3. Trzeciak, A. Synthesis of ‘head-to-tail’ cyclized peptides on solid supports by Fmoc chemistry. Tetrahedron Lett. 33:4557-45560, 1992.

4. Wood, S. J. and Wetzel, R. Novel cyclization chemistry especially suited for biologically derived, unprotected peptides, Int. J. Pept. Protein Res. 39:533-539, 1992.

5. Gilon, C., Halle, D., Chorev, M., Selinger, Z. and Byk, G. Backbone cyclization: A new method for conferring conformational constraint on peptides. Biopolymers 31:745-750, 1991.

6. McMurray, J. S. Solid phase synthesis of a cyclic peptide using Fmoc chemistry. Tetrahedron Letters 32:7679-7682, 1991.

7. Rovero, P. Synthesis of cyclic peptides on solid support. Tetrahedron Letters 32:2639-2642, 1991.

8. Yajima, X. Cyclization on the bead via following Cys Acm deprotection. Tetrahedron 44:805, 1988.

Peptoid

A peptoid is an analogue of a peptide in which one or more of the peptide bonds (NHCO) are replaced by pseudopeptide bonds, which may be the same or different.

Such pseudopeptide bonds may be:

Carba Ψ(CH₂—CH₂)

Depsi Ψ(CO—O)

Hydroxyethylene Ψ(CHOH—CH₂)

Ketomethylene Ψ(CO—CH₂)

Methylene-ocy CH₂—O—

Reduced CH₂—NH

Thiomethylene CH₂—S—

Thiopeptide CS—NH

N-modified —NRCO— (where N is cyclic, branched or linear alkyl of up to 12 carbons)

See also

1. Corringer, P. J., Weng, J. H., Ducos, B., Durieux, C., Boudeau, P., Bohme, A. and Roques, B. P. CCK-B agonist or antagonist activities of structurally hindered and peptidase-resistant Boc-CCK₄ derivatives. J. Med. Chem. 36:166-172, 1993. Amino acids reported: aromatic naphthylalaninimide (Nal-NH2); N-methyl amino acids.

2. Beylin, V. G., Chen, H. G., Dunbar, J., Goel, O. P., Harter, W., Marlatt, M. and Topliss, J. G. Cyclic derivatives of 3,3-diphenylalanine (Dip) (II), novel α-amino acids for peptides of biological interest. Tetrahedron Lett. 34:953-956, 1993.

3. Garbay-Jaureguiberry, C., Ficheux, D. and Roques, B. P. Solid phase synthesis of peptides containing the non-gydrolysable analog of (O)phosphotyrosine, p(CH₂PO₃H₂)Phe. Application to the synthesis of 344-357 sequences of the β₂ adrenergic receptor. Int. J. Pept. Protein Res. 39:523-527, 1992.

4. Luning, B., Norberg, T. and Tejbrant, J. Synthesis of glycosylated amino acids for use in solid phase glycopeptide synthesis, part 2: N-(9-fluorenylmethyloxycarbonyl)-3-O-[2,4,6-tri-O-acetyl-3-O-(2,3,4-tri-O-acetyl-α-D-xylopyranosyl)-β-D-glucopyranosyl]-L]serine. J. Carbohydr. Chem. 11:933-943, 1992.

5. Tung, C. H., Zhu, T., Lackland, H. and Stein, S. An acridine amino acid derivative for use in Fmoc peptide synthesis. Peptide Research 5:115-118, 1992.

6. Eric Frerot, PyBOP and PyBroP: Two reagents for the difficult coupling of the alpha,alpha-dialkyl amino acid Aib. Tetrahedron 47:259-270, 1991.

7. Moree, W. J., Van der Marel, G. A. and Liskamp, R. M. J. Synthesis of peptides containing the β-substituted aminoethane sulfinamide or sulfonamide transition-state isostere derived from amino acids. Tetrahedron Lett. 33:6389-6392, 1992.

8. Rana, T. M. Synthesis of a metal-binding amino acid suitable-for solid phase assembly of peptides. Tetrahedron Lett. 33:4521-4524, 1992.

9. Urge, L., Otvos, L., Jr., Lang, E., Wroblewski, K., Laczko, I. and Hollosi, M. Fmoc-protected, glycosylated asparagines potentially useful as reagents in the solid-phase synthesis of N-glycopeptides. Carbohydr. Res. 235:83-93, 1992.

10. Pavone, V., DiBlasio, B., Lombardi, A., Maglio, O., Isernia, D., Pedone, C., Benedette, E., Altmann, E. and Mutter, M. Non coded C^{α,α}-disubstituted amino acids. X-ray diffraction analysis of a dipeptide containing (S)-α-methylserine. Int. J. Pept. Protein Res. 41:15-20, 1993.

11. Nishino, N., Mihara, H., Kiyota, H., Kobata, K. and Fujimoto, T. Aminoporphyrinic acid as a new template for polypeptide design. J. Chem. Soc. Chem. Commun. 1993:162-163, 1993.

12. Sosnovsky, G., Prakash, I. and Rao, N. U. M. In the search for new anticancer drugs. XXIV: Synthesis and anticancer activity of amino acids and dipeptides containing the 2-chloroethyl- and [N′-nitroso]-aminocarbonyl groups. J. Pharm. Sci. 82:1-10, 1993.

13. Berti, F., Ebert, C. and Gardossi, L. One-step stereospecific synthesis of α,β-dehydroamino acids and dehydropeptides. Tetrahedron Lett. 33:8145-8148, 1992.

Peptidomimetic

A peptidomimetic is a molecule which mimics the biological activity of a peptide, by substantially duplicating the pharmacologically relevant portion of the conformation of the peptide, but is not a peptide or peptoid as defined above. Preferably the peptidomimetic has a molecular weight of less than 700 daltons.

Designing a peptidomimetic usually proceeds by:

(a) identifying the pharmacophoric groups responsible for the activity;

(b) determining the spatial arrangements of the pharmacophoric groups in the active conformation of the peptide; and

(c) selecting a pharmaceutically acceptable template upon which to mount the pharmacophoric groups in a manner which allows them to retain their spatial arrangment in the active conformation of the peptide.

Step (a) may be carried out by preparing mutants of the active peptide and determining the effect of the mutation on activity. One may also examine the 3D structure of a complex of the peptide and the receptor for evidence of interactions, e.g., the fit of a side chain of the peptide into a cleft of the receptor; potential sites for hydrogen bonding, etc.).

Step (b) generally involves determining the 3D structure of the active peptide, in the complex, by NMR spectroscopy or X-ray diffraction studies. The initial 3D model may be refined by an energy minimization and molecular dynamics simulation.

Step (c) may be carried out by reference to a template database, see Wilson, et al. Tetrahedron, 49:3655-63 (1993). The templates will typically allow the mounting of 2-8 pharmacophores, and have a relatively rigid structure. For the latter reason, aromatic structures, such as benzene, biphenyl, phenanthrene and benzodiazepine, are preferred. For orthogonal protection techniques, see Tuchscherer, et al., Tetrahedron, 17:3559-75 (1993).

For more information on peptoids and peptidomimetics, see U.S. Pat. No. 5,811,392, U.S. Pat. No. 5,811,512, U.S. Pat. No. 5,578,629, U.S. Pat. No. 5,817,879, U.S. Pat. No. 5,817,757, U.S. Pat. No. 5,811,515.

Analogues

Also of interest are analogues of the disclosed peptides, and other compounds with activity of interest.

Analogues may be identified by assigning a hashed bitmap structural fingerprint to the compound, based on its chemical structure, and determining the similarity of that fingerprint to that of each compound in a broad chemical database. The fingerprints are determined by the fingerprinting software commercially distributed for that purpose by Daylight Chemical Information Systems, Inc., according to the software release current as of Jan. 8, 1999. In essence, this algorithm generates a bit pattern for each atom, and for its nearest neighbors, with paths up to 7 bonds long. Each pattern serves as a seed to a pseudorandom number generator, the output of which is a set of bits which is logically ored to the developing fingerprint. The fingerprint may be fixed or variable size.

The database may be SPRESI′95 (InfoChem GmbH), Index Chemicus (ISI), MedChem (Pomona/Biobyte), World Drug Index (Derwent), TSCA93(EPA) May bridge organic chemical catalog (Maybridge), Available Chemicals Directory (MDLIS Inc.), NCI96 (NCI), Asinex catalog of organic compounds (Asinex Ltd.), or IBIOScreen SC and NP (Inter BioScreen Ltd.), or an inhouse database.

A compound is an analogue of a reference compound if it has a daylight fingerprint with a similarity (Tanamoto coefficient) of at least 0.85 to the Daylight fingerprint of the reference compound.

A compound is also an analogue of a reference compound if it may be conceptually derived from the reference compound by isosteric replacements.

Homologues are compounds which differ by an increase or decrease in the number of methylene groups in an alkyl moiety.

Classical isosteres are those which meet Erlenmeyer's definition: “atoms, ions or molecules in which the peripheral layers of electrons can be considered to be identical”. Classical isosteres include

| Monovalents | Bivalents | Trivalents | Tetra | Annular |
|---|---|---|---|---|
| F, OH, NH₂, CH₃ | —O— | —N═ | ═C═ | —CH═CH— |
| | | | ═Si═ | |
| Cl, SH, PH₂ | —S— | —P═ | —N+═ | —S— |
| Br | —Se— | —As— | ═P+═ | —O— |
| i | —Te— | —Sb— | ═As+═ | —NH— |
| | | —CH═ | ═Sb+═ | |

Nonclassical isosteric pairs include —CO— and —SO₂—, —COOH and —SO₃H, —SO₂NH₂ and —PO(OH)NH₂, and —H and —F, —OC(═O)— and C(═O)O—, —OH and —NH₂.

Bacteria

The bacteria which are to be inhibited are pathogens of humans or other animals. They may be obligate or opportunistic pathogens. They may be gram-negative or gram-positive bacteria. The gram-negative bacteria include bacteria of the families Pseudomonadacae, Enterobacteriaceae, Vibrionaceae, Bacteroidaceae, Neisseriaceae and Veillonellaceae, and some Bacillaceae, and the order Chlamydiales. The gram-positive bacteria include bacteria of the families Micrococcaceae, Streptococcaceae, Peptococcaceae, some Bacillaceae, and Lactobacillaceae, and the order Rickettsiales. There are also genera of uncertain affiliation, of which the gram-negative Brucella, Bordetella, Francisella, Chromobacterium, Haemophilus, Pasteurella, Actinobacillus, Cardiobacterium, Streptobacillus, and Calymmatobacterium, and the gram-positive Listeria Erysipelothrix, and Corynebacterium, are worthy of note.

Among the gram-negative bacteria, the Enterobacteriaceae (Escherichia, Edwardsiella, Citrobacter, Salmonella, Shigella, Klebsiella, Enterobacter, Hafnia, Serratia, Proteus, Yersinia, and Erwinia) are of particular interest.

Other Microbes

The peptides of the present invention may also be useful in inhibiting other microbial pathogens, including algal, fungal and protozooal pathogens.

Other Target Organisms

The peptides of the present invention may also be useful in inhibiting nonmicrobial pathogens, such as worms or arthropods, whose membranes are sufficiently different from mammalian membranes. In addition they may be useful as spermicides for humans, as the sperm membrane is atypical of human cell membranes.

Antimicrobial Activity

Preferably, the peptides of the present invention have an antimicrobial activity at least equal to that of magainin 2 under the same assay conditions.

Patients/Subjects

The terms “patients” and “subjects” are used interchangeably. The term “animal” includes “humans”. The subject is preferably a mammal, especially of the orders Primata (humans, apes, monkeys), Artiodactyla or Perissodactyla (esp. cows, pigs, goats, sheep, horses), Rodenta or Lagomorpha (esp. rats, mice, rabbits, hamsters), or Carnivora (esp. cats and dogs) or other pet, farm or laboratory mammals.. It is especially preferable that the subject be human, but the subject may be a nonhuman mammal.

Those peptides which are effective in nonhuman mammals but not humans have the advantage that their animal use does not endanger human antimicrobial therapy.

Pharmaceutical Methods and Preparations

The term “protection”, as used herein, is intended to include “prevention,” “suppression” and “treatment.” “Prevention” involves administration of the protein prior to the induction of the disease (or other adverse clinical condition). “Suppression” involves administration of the composition prior to the clinical appearance of the disease. “Treatment” involves administration of the protective composition after the appearance of the disease.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.” It should also be understood that to be useful, the protection provided need not be absolute, provided that it is sufficient to carry clinical value. An agent which provides protection to a lesser degree than do competitive agents may still be of value if the other agents are ineffective for a particular individual, if it can be used in combination with other agents to enhance the level of protection, or if it is safer than competitive agents. The drug may provide a curative effect, an ameliorative effect, or both.

At least one of the drugs of the present invention may be administered, by any means that achieve their intended purpose, to protect a subject against a disease or other adverse condition. The form of administration may be systemic or topical. For example, administration of such a composition may be by various parenteral routes such as subcutaneous, intravenous, intradermal, intramuscular, intraperitoneal, intranasal, transdermal, or buccal routes. Alternatively, or concurrently, administration may be by the oral route. Parenteral administration can be by bolus injection or by gradual perfusion over time.

A typical regimen comprises administration of an effective amount of the drug, administered over a period ranging from a single dose, to dosing over a period of hours, days, weeks, months, or years. The specific amount of drug administered can be determined readily for any particular patient according to recognized procedures and based on the expertise and experience of the skilled practitioner. Precise dosing for a patient can be determined according to routine medical practice.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The standard dose of a drug will generally be determined by first administering a trial dose to a test animal. This trial dose may be determined by a theoretical calculation (e.g., one based on the binding affinity of the drug for a receptor, and the number of receptors in the body), or by analogy with a related drug for which a safe and effective dose is known. The dose is then adjusted upward if the initial dose is safe but insufficiently effective, and downward if the initial dose is unsafe. These adjustments may be arithmetic or logarithmic,, and typically progress from coarse to fine. In this manner, a range of doses which are reasonably safe and effective in the test animal is determined.

The initial human dose is then determined on the basis of the preferred dose in one or more test animal species, with suitable adjustments for the differences between the human and the test animal, and usually erring on the side of safety. If the test animal is a generally accepted model of the disease in question, there will be known drugs for which the safe and effective dose in both humans and the test animal in question are known, allowing a conversion of the animal dose to the human dose. If not, the test animal dose will normally be corrected on the basis of the relative weight or surface area of the test animal and the human. This initial human dose is then adjusted in a similar manner to that described for the test animal.

In one embodiment, the initial toxicological testing will be in mice and will involve a maximum dose of lg/kg body weight.

Once a standard dose is determined for the patient population in the abstract, a clinician may determine what further adjustment is appropriate for a particular patient. It is understood that the suitable dosage of a drug of the present invention will be dependent upon the age, sex, health, and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This will typically involve adjustment of a standard dose, e.g., reduction of the dose if the patient has a low body weight.

The total dose required for each treatment may be administered by multiple doses or in a single dose. The total daily dose of a drug for a human adult will typically be in the range of 1 pg to 10 g, more typically in the range of 1 ng to 1 g, still more typically in the range of 1 μg to 1 g, most typically in the range of 1 mg to 1 g.

The drug may be administered alone or in conjunction with other therapeutics directed to the disease or directed to other symptoms thereof. Two drugs are administered in “conjunction” if their times of administration are sufficiently close so that (1) one drug alters the biological response to the other drug, or (2) both drugs have a protective effect on the subject at the same time.

The appropriate dosage form will depend on the disease, the protein, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

In the case of peptide drugs, the drug may be administered in the form of an expression vector comprising a nucleic acid encoding the peptide, such a vector, after in corporation into the genetic complement of a cell of the patient, directs synthesis of the peptide. Suitable vectors include genetically engineered poxviruses (vaccinia), adenoviruses, adeno-associated viruses, herpesviruses and lentiviruses which are or have been rendered nonpathogenic. Alternatively, a nonpathogenic bacterium could be genetically engineered to express the drug. The drug must, of course, either be secreted, or displayed on the outer membrane of the bacterium (or the coat of a virus) in such a manner that it can interact with the appropriate receptor. The dose of vector will be sufficient to achieve a suitable expressed and delivered dose of the peptide drug as previously discussed. Since vectors replicate, and peptides are continually manufactured by the transformed cells (at least when the corresponding promoter is active), it is possible to achieve a clinical effect with a relatively small amount of the vector.

In addition to at least one drug as described herein, a pharmaceutical composition may contain suitable pharmaceutically acceptable carriers, such as excipients, carriers and/or auxiliaries which facilitate processing of the active compounds into preparations which can be used pharmaceutically. See, e.g., Berker, supra, Goodman, supra, Avery, supra and Ebadi, supra, which are entirely incorporated herein by reference, included all references cited therein.

The appropriate dosage form depends on the status of the disease, the composition administered, and the route of administration. Dosage forms include tablets, capsules, lozenges, dental pastes, suppositories, inhalants, solutions, ointments, and parenteral depots. See, e.g., Berker, supra, Goodman, supra, Avery, sudra and Ebadi, supra, which are entirely incorporated herein by reference, including all references cited therein.

In one embodiment, the drug is dissolved or suspended in an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

For solid compositions, conventional nontoxic solid-carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the drugs are preferably supplied in finely divided form along with a surfactant and propellant. Typical percentages of drugs are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms,. such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. the balance of the composition is ordinarily propellant. A carrier can also be included, as desired, as with, e.g., lecithin for intranasal delivery.

Specific Diagnostic Uses

The peptides of the present invention are of particular interest in localizing an infection or detecting sepsis. Technetium-labeled peptides could be used to specifically locate bacteria in vivo using imaging.

Binding Molecule

For the purpose of the discussion of diagnostic methods and agents which follows, the “binding molecule” may be a peptide, peptoid or peptidomimetic of the present invention, or an oligonucleotide of the present invention, which binds the analyte or a binding partner of the analyte. The analyte is a target protein.

In Vitro Diagnostic Methods and Reagents

The in vitro assays of the present invention may be applied to any suitable analyte-containing sample, and may be qualitative or quantitative in nature. In order to detect the presence, or measure the amount, of an analyte, the assay must provide for a signal producing system (SPS) in which there is a detectable difference in the signal produced, depending on whether the analyte is present or absent (or, in a quantitative assay, on the amount of the analyte). The detectable signal may be one which is visually detectable, or one detectable only with instruments. Possible signals include production of colored or luminescent products, alteration of the characteristics (including amplitude or polarization) of absorption or emission of radiation by an assay component or product, and precipitation or agglutination of a component or product. The term “signal” is intended to include the discontinuance of an existing signal, or a change in the rate of change of an observable parameter, rather than a change in its absolute value. The signal may be monitored manually or automatically.

The component of the signal producing system which is most intimately associated with the diagnostic reagent is called the “label”. A.label may be, e.g., a radioisotope, a fluorophore, an enzyme, a co-enzyme, an enzyme substrate, an electron-dense compound, an agglutinable particle. One diagnostic reagent is a conjugate, direct or indirect, or covalent or noncovalent, of a label with a binding molecule of the invention.

The radioactive isotope can be detected by such means as the use of a gamma counter or a scintillation counter or by autoradiography. Isotopes which are particularly useful for the purpose of the present invention are ³H, ¹²⁵I, ¹³¹I, ³⁵S, ¹⁴C, and, preferably, ¹²⁵I.

It is also possible to label a compound with a fluorescent compound. When the fluorescently labeled antibody is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

Alternatively, fluorescence-emitting metals such as ¹²⁵Eu, or others of the lanthanide series, may be attached to the binding protein using such metal chelating groups as diethylenetriaminepentaacetic acid (DTPA) of ethylenediamine-tetraacetic acid (EDTA).

The binding molecules also can be detectably labeled by coupling to a chemiluminescent compound. The presence of the chemiluminescent compound is then determined by detecting the presence of luminescence that arises during the course of a chemical reaction after a.suitable reactant is provided. Examples of particularly useful chemiluminescent labeling compounds are luminol, isolumino, theromatic acridinium ester, imidazole, acridinium salt and oxalate ester.

Likewise, a bioluminescent compound may be used to label the binding molecule. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Enzyme labels, such as horseradish peroxidase and alkaline phosphatase, are preferred. When an enzyme label is used, the signal producing system must also include a substrate for the enzyme. If the enzymatic reaction product is not itself detectable, the SPS will include one or more additional reactants so that a detectable product appears.

Assays may be divided into two basic types, heterogeneous and homogeneous. In heterogeneous assays, the interaction between the affinity molecule and the analyte does not affect the label, hence, to determine the amount or presence of analyte, bound label must be separated from free label. In homogeneous assays, the interaction does affect the activity of the label, and therefore analyte levels can be deduced without the need for a separation step.

In general, a target-binding molecule of the present invention may be used diagnostically in the same way that a target-binding antibody is used. Thus, depending on the assay format, it may be used to assay the target, or by competitive inhibition, other substances which bind the target. The sample will normally be a biological fluid, such as blood, urine, lymph, semen, milk, or cerebrospinal fluid, or a fraction or derivative thereof, or a biological tissue, in the form of, e.g., a tissue section or homogenate. However, the sample conceivably could be (or derived from) a food or beverage, a pharmaceutical or diagnostic composition, soil, or surface or ground water. If a biological fluid or tissue, it may be taken from a human or other mammal, vertebrate or animal, or from a plant. The preferred sample is blood, or a fraction or derivative thereof.

In one embodiment, the binding molecule is insolubilized by coupling it to a macromolecular support, and target in the sample is allowed to compete with a known quantity of a labeled or specifically labelable target analogue. (The conjugate of the binding molecule to a macromolecular support is another diagnostic agent within the present invention.) The “target analogue” is a molecule capable of competing with target for binding to the binding molecule, and the term is intended to include target itself. It may be labeled already, or it may be labeled subsequently by specifically binding the label to a moiety differentiating the target analogue from authentic target. The solid and liquid phases are separated, and the labeled target analogue in one phase is quantified. The higher the level of target analogue in the solid phase, i.e., sticking to the binding molecule, the lower the level of target analyte in the sample.

In a “sandwich assay”, both an insolubilized target-binding molecule, and a labeled target-binding molecule are employed. The target analyte is captured by the insolubilized target-binding molecule and is tagged by the labeled target-binding molecule, forming a tertiary complex. The reagents may be added to the sample in either order, or simultaneously. The target-binding molecules may be the same or different, and only one need be a target-binding molecule according to the present invention (the other may be, e.g., an antibody or a specific binding fragment thereof). The amount of labeled target-binding molecule in the tertiary complex is directly proportional to the amount of target analyte in the sample.

The two embodiments described above are both heterogeneous assays. However, homogeneous assays are conceivable. The key is that the label be affected by whether or not the complex is formed.

A label may be conjugated, directly or indirectly (e.g., through a labeled anti-target-binding molecule antibody), covalently (e.g., with SPDP) or noncovalently, to the target-binding molecule, to produce a diagnostic reagent. Similarly, the target binding molecule may be conjugated to a solid-phase support to form a solid phase (“capture”) diagnostic reagent. Suitable supports include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material may have virtually any possible structural configuration so long as the coupled molecule is capable of binding to its target. Thus the support configuration may be spherical, as in a bead, or cylindrical, as in the inside surface of a test tube, or the external surface of a rod. Alternatively, the surface may be flat such as a sheet, test strip, etc.

In Vivo Diagnostic Uses

Analyte-binding molecules can be used for in vivo imaging.

Radio-labelled binding molecule may be administered to the human or animal subject. Administration is typically by injection, e.g., intravenous or arterial or other means of administration in a quantity sufficient to permit subsequent dynamic and/or static imaging using suitable radio-detecting devices. The preferred dosage is the smallest amount capable of providing a diagnostically effective image, and may be determined by means conventional in the art, using known radio-imaging agents as a guide.

Typically, the imaging is carried out on the whole body of the subject, or on that portion of the body or organ relevant to the condition or disease under study. The radio-labelled binding molecule has accumulated. The amount of radio-labelled binding molecule accumulated at a given point in time in relevant target organs can then be quantified.

A particularly suitable radio-detecting device is a scintillation camera, such as a gamma camera. A scintillation camera is a stationary device that can be used to image distribution of radio-labelled binding molecule. The detection device in the camera senses the radioactive decay, the distribution-of which can be recorded. Data produced by the imaging system can be digitized. The digitized information can be analyzed over time discontinuously or continuously. The digitized data can be processed to produce images, called frames, of the pattern of uptake of the radio-labelled binding protein in the target organ at a discrete point in time. In most continuous (dynamic) studies, quantitative data is obtained by observing changes in distributions of radioactive decay in target organs over time. In other words, a time-activity analysis of the data will illustrate uptake through clearance of the radio-labelled binding molecule by the target organs with time.

Various factors should be taken into consideration in selecting an appropriate radioisotope. The radioisotope must be selected with a view to obtaining good quality resolution upon imaging, should be safe for diagnostic use in humans and animals, and should preferably have a short physical half-life so as to decrease the amount of radiation received-by the body. The radioisotope used should. preferably be pharmacologically inert, and, in the quantities administered, should not have any substantial physiological effect.

The binding molecule may be radio-labelled with different isotopes of iodine, for example ¹²³I, ¹²⁵I, or ¹³¹I (see for example, U.S. Pat. No. 4,609,725). The extent of radio-labeling must, however be monitored, since it will affect the calculations made based on the imaging results (i.e. a diiodinated binding molecule will result in twice the radiation count of a similar monoiodinated binding molecule over the same time frame).

In applications to human subjects, it may be desirable to use radioisotopes other than ¹²⁵I for labelling in order to decrease the total dosimetry exposure of the human body and to optimize the detectability of the labelled molecule (though this radioisotope can be used if circumstances require). Ready availability for clinical use is also a factor. Accordingly, for human applications, preferred radio-labels are for example, ^{99m}Tc, ⁶⁷Ga, ⁶⁸Ga, ⁹⁰Y, ¹¹¹In, ^{113m}In , ¹²³I, ¹⁸⁶Re, ¹⁸⁸Re or ²¹¹At.

The radio-labelled binding molecule may be prepared by various methods. These include radio-halogenation by the chloramine—T method or the lactoperoxidase method and subsequent purification by HPLC (high pressure liquid chromatography), for example as described by J. Gutkowska et al in “Endocrinology and Metabolism Clinics of America: (1987) 16 (1):183. Other known method of radio-labelling can be used, such as IODOBEADS™.

There are a number of different methods of delivering the radio-labelled binding molecule to the end-user. It may be administered by any means that enables the active agent to reach the agent's site of action in the body of a mammal. If the molecule is digestible when administered orally, parenteral administration, e.g., intravenous, subcutaneous, or intramuscular, would ordinarily be used to optimize absorption.

Other Uses

The binding molecules of the present invention may also be used to purify target from a fluid, e.g., blood. For this purpose, the target-binding molecule is preferably immobilized on a solid-phase support. Such supports include those already mentioned as useful in preparing solid phase diagnostic reagents.

Peptides, in general, can be used as molecular weight markers for reference in the separation or purification of peptides by electrophoresis or chromatography. In many instances, peptides may need to be denatured to serve as molecular weight markers. A second general utility for peptides is the use of hydrolyzed peptides as a nutrient source. Hydrolyzed peptide are commonly used as a growth media component for culturing microorganisms, as well as a food ingredient for human consumption. Enzymatic or acid hydrolysis is normally carried out either to completion, resulting in free amino acids, or partially, to generate both peptides and amino acids. However, unlike acid hydrolysis, enzymatic hydrolysis (proteolysis) does not remove non-amino acid functional groups that may be present. Peptides may also be used to increase the viscosity of a solution.

The peptides of the present invention may be used for any of the foregoing purposes, as well as for therapeutic and diagnostic purposes as discussed further earlier in this specification.

### REFERENCE EXAMPLE A

### Determination of Peptide Conformation

One method of determining the conformation of a peptide when bound to lipids is illustrated below, using the A₁₉ analogue of magainin 2 (Ala₁₉-magainin) as an example. We used both Fourier transform infrared (FTIR) and solid-state nuclear magnetic resonance (NMR) spectroscopy.

The peptide was synthesized with both ¹³C and ¹⁵N labels in order to determine conformation through intramolecular distance measurements using rotational-echo double resonance (REDOR) NMR.³² In these studies, we attempted to (a) determine whether or not magainins are completely α-helical when bound to membranes, (b) localize secondary structures within the molecule, and (c) identify the location of the peptide with respect to the lipid bilayer.²²

The amide I band in the infrared spectrum is sensitive to peptide conformation. This band was centered near 1650 cm⁻¹ with a prominent shoulder near 1635 cm⁻¹. The former frequency is attributable to α-helical conformation, while the latter is generally associated with β-sheet structure.³³ For fully hydrated lipid-peptide mixtures, we estimated that the α-helical and β-sheet components of the amide I′ band (where amide protons are exchanged with deuterium) account for 60-75% and 20-35%, respectively, of the total peak area, depending on the fluidity of the lipids. The level of hydration of the system is important in determining the secondary structure of the peptide. In the anhydrous state, the peptide is almost entirely-α-helical. As hydration occurred (using D₂O as the solvent), the helical content diminished, with a concomitant increase in β-sheet structure. Also, the quick disappearance of the amide II band upon hydration with D₂O showed that the peptide molecules have access to the aqueous surroundings and are not locked in hydrogen bonds that would impede the exchange of deuite-rium for hydrogen along the peptide backbone. Other investigators also saw a mixture of α-helix and β-sheet or turn structures in membrane-bound magainins using CD,^{19, 25} FTIR,³⁴ and Raman³⁵ spectroscopy.

In order to confirm the presence of both α-helical and β-sheet structure in Alalg-magainin, we synthesized the peptide with [1-¹³C]Ala₁₅, and [¹⁵N]Ala₁₉. This labeling pattern allows for a distance measurement between Ala₁₅ and Ala₁₉ by using REDOR to detect dipolar coupling between the ¹³C and ¹⁵N nuclei. If the peptide adopts an α-helical conformation, these atoms will be only 4 Å apart. The ¹³C NMR spectrum showed two peaks at 176.8 and 172.4 ppm for [1-¹³C]Ala₁₅, which is consistent with a mixture of α-helix and β-sheet conformations. Furthermore, only the peak at 176.8 ppm showed dipolar coupling with ¹⁵N in the REDOR experiment, confirming its assignment as the α-helical component. By comparing the areas of the two peaks, the proportion of α-helix to β-sheet structure using solid-state NMR was nearly identical to the results obtained by FTIR spectroscopy. Thus, we concluded that while the peptide is primarily α-helical, there is a substantial amount of β-sheet structure as well.

We also attempted to determine the location of the peptide in the lipid bilayer using ¹³C-observe, ³¹P-dephase REDOR experiment. If the peptide resides close to the lipid polar head groups, the distance between the ¹³C label in the peptide and ³¹P in the lipid molecules should be short, resulting in significant dipolar coupling. The results indicated that most of the peptide must reside near the surface of the bilayer, although it was not possible to rule out that some of the peptide might penetrate through the hydrophobic core of the bilayer in channels or pores.

### EXAMPLE 1

PGLa contains GAIAGKIAK (residues 7-15 of SEQ ID NO:2) As shown in Table I, the activity of this peptide is very high and it also possesses low hemolytic activity. This peptide is capable of forming a highly amphipathic α-helix. We wanted to test the hypothesis that the ability to form a highly amphipathic α-helix is a mandatory requirement for potent antimicrobial activity in these linear cationic peptides. The placement of lysines at positions 1 and 5 of a heptamer repeat results in their clustering on the helical face of a 21-mer peptide (SEQ ID NO:4). We decided to shift a lysine from position 5 to 7 of the heptamer. The resulting peptide, (KLAGLAK)₃—NH₂ (SEQ ID NO:6), has a considerably reduced hydrophobic moment as an α-helix because the lysines now are split into two clusters of three on the helical face. Finally, we designed a third peptide, (KIGAKI)₃-NH₂ (SEQ ID NO:8), that would have no propensity to form an amphipathic helix, but could form a highly amphipathic β-sheet. All three of these peptides carry a +7 charge and are nearly equal in overall hydrophobicities. FIG. 1 shows helical wheel and β-sheet diagrams for the peptides, along with the calculated hydrophobic moments for each of the structures.

If forming an amphipathic structure is critical to membrane association, (KIAGKIA)₃-NH₂ (SEQ ID NO:4) should be effective primarily as an α-helix, (KLAGLAK)₃—NH₂ (SEQ ID NO:6) is only slightly more amphipathic as an α-helix than as a β-sheet, and KIGAKI)₃-NH₂ (SEQ ID NO:8) is overwhelmingly amphipathic as a β-sheet. Our plan was to compare the antimicrobial and hemolytic activities of the peptides, their conformations when bound to lipid bilayers, and their ability to induce leakage from LUV.

Experimental Procedures

Materials. All peptides were synthesized using Fmoc chemistry on an Advanced Chem Tech model 90 peptide synthesizer. The crude peptides were purified by reverse phase HPLC. Purity was checked by reverse phase HPLC, capillary electrophoresis, and electrospray mass spectrometry. POPC, POPE, POPG, and E. coli polar lipid extract were used as supplied from Avanti Polar Lipids, Inc. E. coli DPG, calcein, TFE, and buffer materials were from Sigma Chemical Co. Phosphorus content in lipid stock solutions was determined by a spectrophotometric analysis (39).

Antimicrobial and Hemolytic Assays. Antimicrobial susceptibility testing against Staphylococcus aureus (ATCC 29213), Escherichia coli (ATCC 25922), and Pseudomonas aeruginosa (ATCC 27853) was performed using a modification of the National Committee for Clinical Laboratory Standards microdilution broth assay (22). Mueller-Hinton broth (BBL) was used for diluting the peptide stock solution and for diluting the bacterial inoculum. The inoculum was prepared from mid-logarithmic phase cultures. Microtiter plate wells received aliquots-of 100 μL each of the inoculum and peptide dilution. The final concentration of peptide solution ranged-from 0.25 to 256 μg/mL in two-fold dilutions. The final concentration of bacteria in the wells was 5×10⁵ CFU/mL. Peptides were tested in duplicate. In addition to the test peptide, three standard peptides and a non-treated growth control were included to validate the assay. The microtiter plates were incubated overnight at 37° C. and the absorbance was measured at 600 nm. MIC is defined as the lowest concentration of peptide that completely inhibits growth of the organism. Hemolysis at peptide concentrations of 500 μg/mL was determined using a 5% suspension of freshly drawn human erythrocytes, which had been washed twice in phosphate buffered saline. After incubation at 37° C. for 30 min, the suspension was centrifuged at 10,000×g for 10 min and the absorbance at 400 nm was measured. Complete hemolysis was determined by adding 0.2% Triton X-100 in place of the peptide.

CD Spectroscopy. CD spectra were measured using a Jasco J-715 spectropolarimeter. Spectra were recorded from 250-190 nm at a sensitivity of 100 mdeg, resolution of 0.1 nm, response of 8 seconds, bandwidth of 1.0 nm, and scan speed of 20 nm/min, with a single accumulation. The buffer contained 10 mM potassium phosphate, 150 mM KCl, 1 mM EDTA, pH 7.0. The peptide concentration in buffer and TFE/buffer mixtures was 20 μM. LUV were prepared from aqueous dispersions of POPG at a concentration of ˜1 mg/mL in phosphate buffer. Following five freeze/thaw cycles, the mixture was extruded ten times through a 0.1-μm-pore polycarbonate membrane in an Avanti mini-extruder apparatus, resulting in ˜100-nm-diameter LUV. The lipid and peptide concentrations in the cuvet were 100 and 5 μM, respectively.

FTIR Spectroscopy. Mixtures of POPG (4 μmoles) and peptide (0.2 μmoles) were co-dissolved in 2:1 CHCl₃/CH₃OH. Solvent was removed by evaporation, followed by evacuation under high vacuum. The mixtures were suspended in D₂O buffer (20 mM PIPES, 1 mM EGTA, pD 7.0), isolated by centrifugation, and placed between CaF₂ windows using a 25 μm Teflon spacer. FTIR spectra were collected using a Mattson Polaris FTIR spectrometer with a HgCdTe detector. A total of 250 interferograms were co-added and Fourier transformed with triangular apodization to generate absorbance spectra with 2 cm⁻¹ resolution and data points encoded every 1 cm⁻¹, with a signal-to-noise ratio of better than 500(22).

Peptide-Induced Leakage from Calcein-Loaded LUV. The ability of the peptides to release calcein (MW 623) from LUV of varying lipid composition was compared. LUV were prepared as above, except that-the buffer consisted of 50 mM HEPES, 100 mM NaCl, 0.3 mM EDTA, 80 mM calcein, pH 7.4. Calcein-loaded vesicles were separated from free calcein by size-exclusion chromatography using a Sephadex G-50 column and calcein-free buffer. Calcein leakage was monitored using a Perkin-Elmer LS-500 luminescence spectrometer by measuring the time-dependent increase in fluorescence of calcein (excitation=490 nm, emission=520 nm). LUV containing 8 nmoles of lipid were added to 1.5 mL of buffer in a stirred cuvet at 25° C. An aliquot of peptide was added to achieve the desired lipid-to-peptide ratio (256 to 8). Complete leakage was determined by the addition of 20 μL of 10% Triton X-100. Each value represents at least nine different measurements using at least three different LUV preparations.

Fluorescence Measurement of Peptide Binding to LUV. Peptide binding to LUV was assessed by measuring the shift in the emission maximum of the tryptophan residue in W-KIAGKIA, W-KLAGLAK, and W-KIGAKI. LUV were prepared as described for the leakage experiments except that calcein was omitted. Fluorescence spectra were collected from 290 to 400 nm using an excitation wavelength of 280 nm. The peptide concentration was kept constant at 3 μM.

DSC. Lipid films were made by solvent evaporation under nitrogen of solutions of DiPoPE in chloroform/methanol (2/1,v/v). Last traces of solvent were removed in vacuum for 2 hours. The films were then hydrated by vortexing at room temperature with 20 mM PIPES, 1 mM EDTA, 150 mM NaCl with 0.002% NaN₃, pH 7.4. The final lipid concentration was 3.7 mg/mL. The lipid suspension was degassed under vacuum before being loaded into a NanoCal high sensitivity scanning calorimeter (CSC, Provo, Utah). A heating scan rate of 0.75° C./min was generally employed. T_{H} was fitted using parameters to describe equilibrium with a single van't Hoff enthalpy and the transition temperature reported as that for the fitted curve. Data was analysed with the program Origin, version 5.0.

Results

Comparison of Antimicrobial and Hemolytic Activities of the Peptides. The antimicrobial and hemolytic activities of the three model peptides are compared to those of magainin 2 amide and PGLa in Table II. KIAGKIAx3, KLAGLAKx3, and KIGAKIx3 are significantly more potent against all three microorganisms than either magainin 2 amide or PGLa. At 500 μg/mL, all of the peptides tested showed little hemolytic activity.

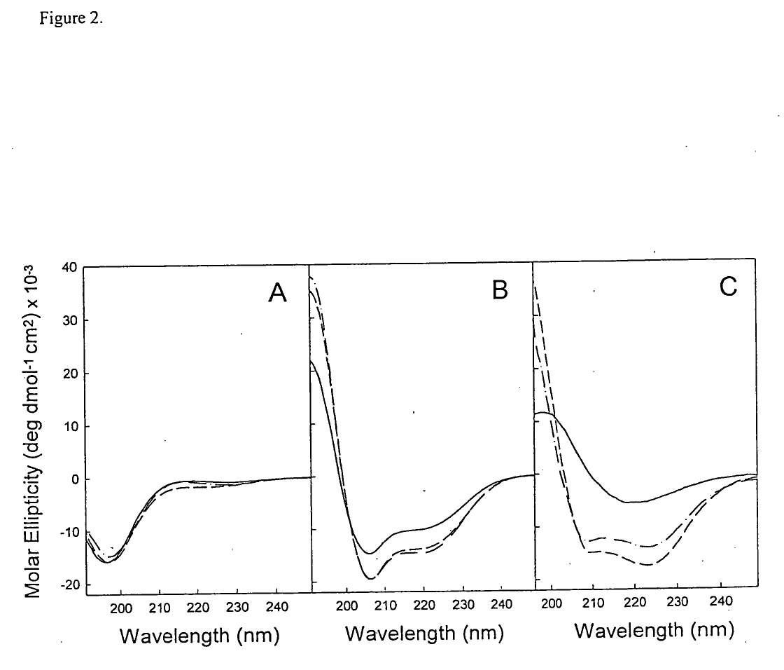

Secondary Structure of the Peptides. The conformation of the peptides was assessed by CD and FTIR spectroscopy. As shown in FIG. 2, the CD spectra of the three model peptides are characteristic of a random structure (with a minimum below 200 nm) in buffer (panel A). TFE is often used as a membrane-mimetic to lower the polarity of the solvent. In 50% TFE (panel B), the spectra show minima near 208 and 222 nm, which indicates α-helical content. The amount of helical structure in KIAGKIAx3 and KLAGLAKx3 is about the same, while the helical content of KIGAKIx3 is slightly lower under these conditions. Finally, spectra of the peptides in the presence of POPG LUV (lipid-to-peptide ratio of 20:1) reveal that both KIAGKIAx3 and KLAGLAKx3 are mainly α-helical, with the helical content of KIAGKIAx3 slightly greater than that of KLAGLAKx3. The conformation of KIGAKIx3 is dramatically different in the presence of POPG vesicles as compared to TFE. This spectrum, with a single minimum just below 220 nm, suggests β-sheet structure(20).

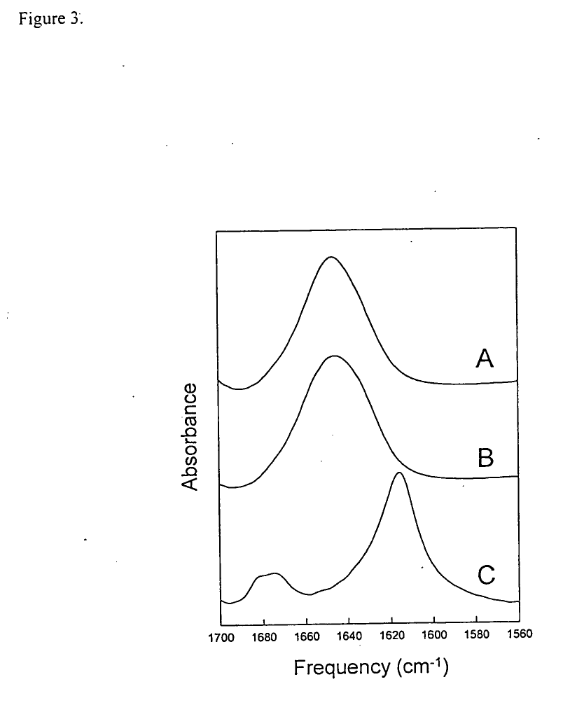

The amide I′ vibrational bands of the three peptides bound to POPG at a 20:1 molar ratio of lipid to peptide are shown in FIG. 3. The amide I′ band for KIAGKIAx3 is centered close to 1650 cm⁻¹, indicative of primarily α-helical conformation. In contrast, the band for KLAGLAKx3 is shifted slightly to lower frequency and is broader, suggesting less α-helical content, in agreement with the CD data. The amide I′ band for KIGAKIx3 is markedly different, with a maximum below 1620 cm⁻¹ and a small peak near 1680 cm⁻¹, consistent with β-sheet conformation (51).

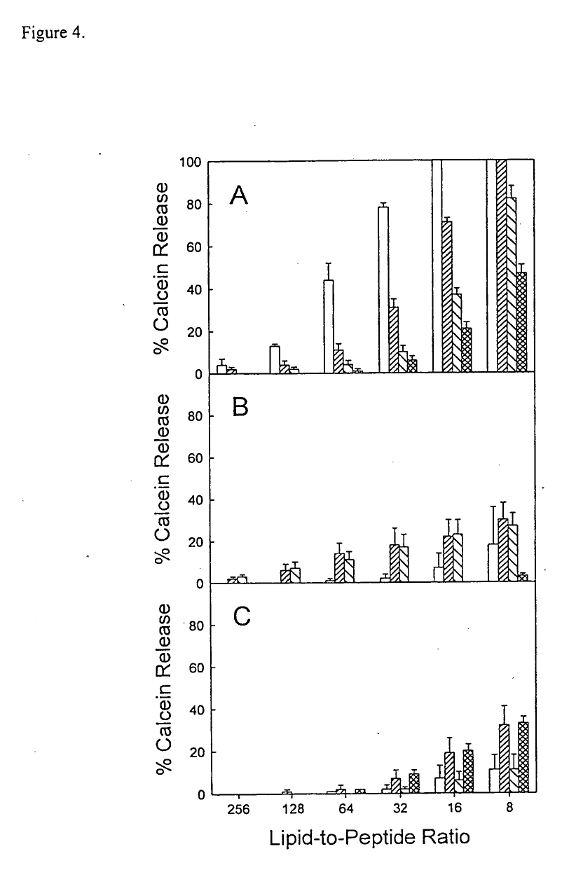

Peptide-Induced Leakage from Cal cein-Loaded LUV. A comparison of PGLa and the three model peptides in their ability to release calcein from LUV with varying lipid composition is shown in FIG. 4. All peptides were able to induce leakage from anionic LUV composed of POPG (panel A). The order of potency is: PGLa>KIAGKIAx3>KLAGLAKx3>KIGAKIx3. Thus, PGLa, the peptide with the weakest antimicrobial activity, is the most potent in inducing calcein release. In POPC LUV (panel B), the level of calcein release was much lower than in POPG LUV; however, it is clear that the three α-helical peptides (PGLa, KIAGKIAx3, and KLAGLAKx3) were more active than KIGAKI in permeabilizing POPC vesicles.

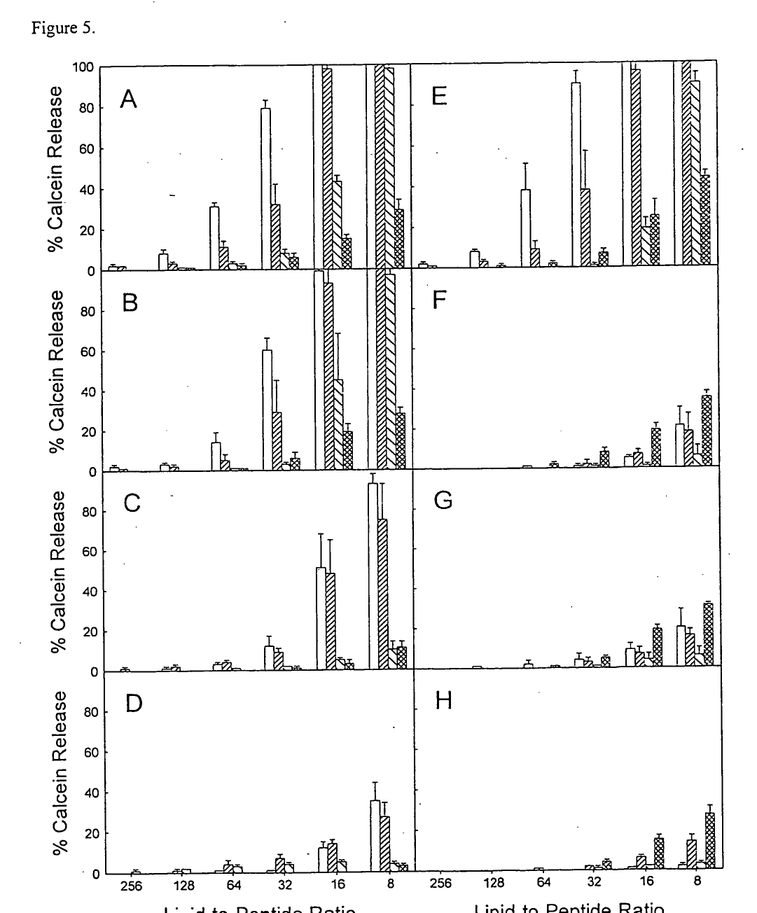

In order to better relate the leakage experiments to bacterial membranes, calcein-loaded LUV composed of E. coli polar lipids were tested. These results, shown in panel C, are dramatically different. The order of potency here is: KIGAKIx3=KIAGKIAx3>KLAGLAKx3=PGLa. The correlation between the antimicrobial activity and leakage in E. coli LUV is much better than in POPG or POPC LUV. The composition of E. coli polar lipids is as follows: 67% PE, 23% PG, and 10% DPG. Therefore, we decided to compare leakage rates in LUV containing POPG as the anionic component and either POPC or POPE as the neutral component. The ability of the peptides to increase the permeability of LUV with neutral-to-acidic lipid ratios of 1:1, 2:1, 3:1, and 4:1 is shown in FIG. 5. The general trend is a reduction in leakage as the neutral-to-acidic lipid ratio increases. In POPC/POPG LUV (panels A-D), PGLa is the most potent peptides at all ratios, followed closely by KIAGKIAx3. The lytic activity of KLAGLAKx3 falls off dramatically at higher neutral lipid content. KIGAKIx3 is the least potent peptide at all ratios. The results change markedly, however, when POPC is replaced by POPE (panels E-H). At 2:1 POPE/POPG, leakage rates for the three α-helical peptides decrease significantly, while the activity of KIGAKI is slightly enhanced in comparison to 2:1 POPC/POPG. By 4:1 POPE/POPG, the activity of PGLa is quite low even at high peptide levels. KIAGKIAx3 and KIGAKIx3 are the most potent, with leakage rates only slightly below those observed in E. coli LUV.

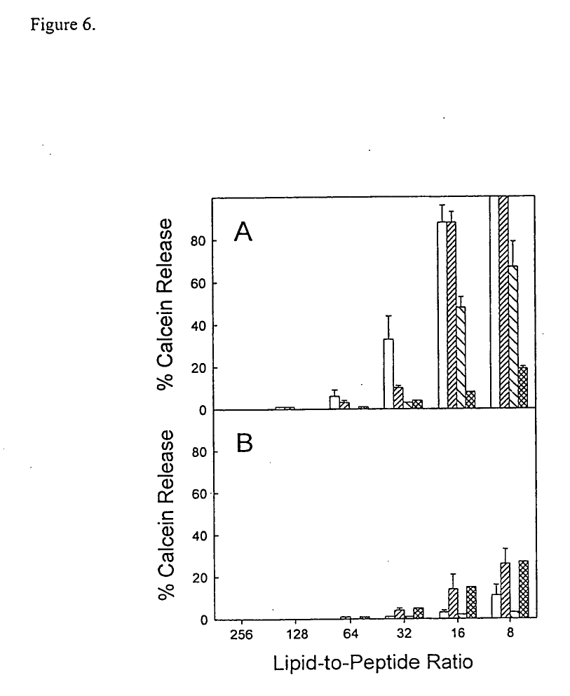

Since E. coli plasma membrane lipids contain DPG as a third major lipid component, LUV were prepared with a ternary mixture of 23% POPG, 10% DPG, and 67% either POPE or POPC (FIG. 6). LUV containing POPC as the neutral lipid (panel A) are highly susceptible to the lytic activity of the α-helical peptides but not the β-sheet peptide. In contrast, the properties of LUV formed by combining POPE, POPG, and DPG (panel B) are quite similar to those of LUV made from the E. coli lipid extract (FIG. 4, C). Only the activity of KIGAKIx3 is enhanced by replacing POPC with POPE in the ternary mixture.

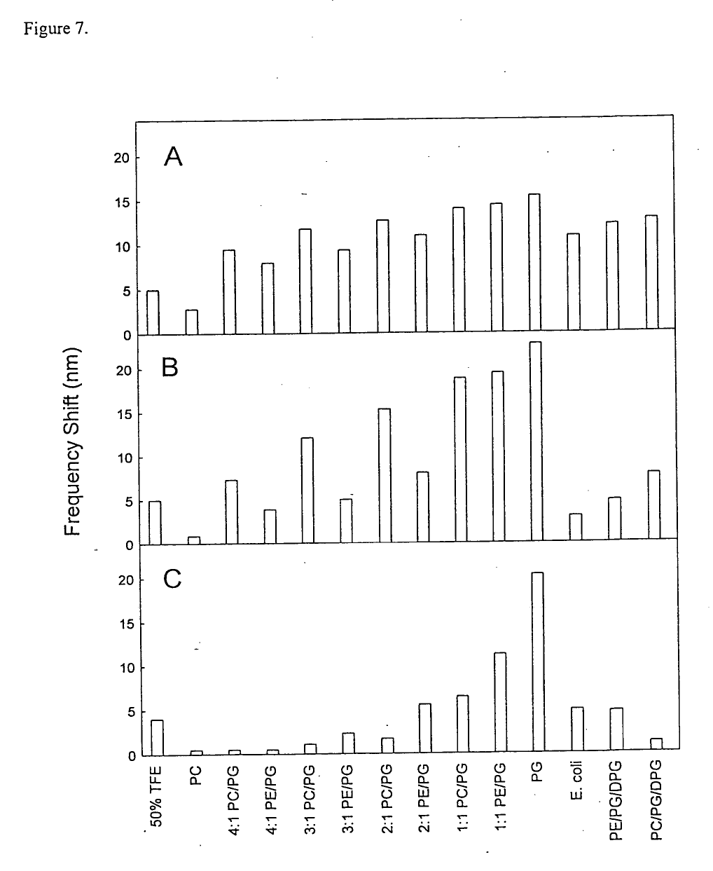

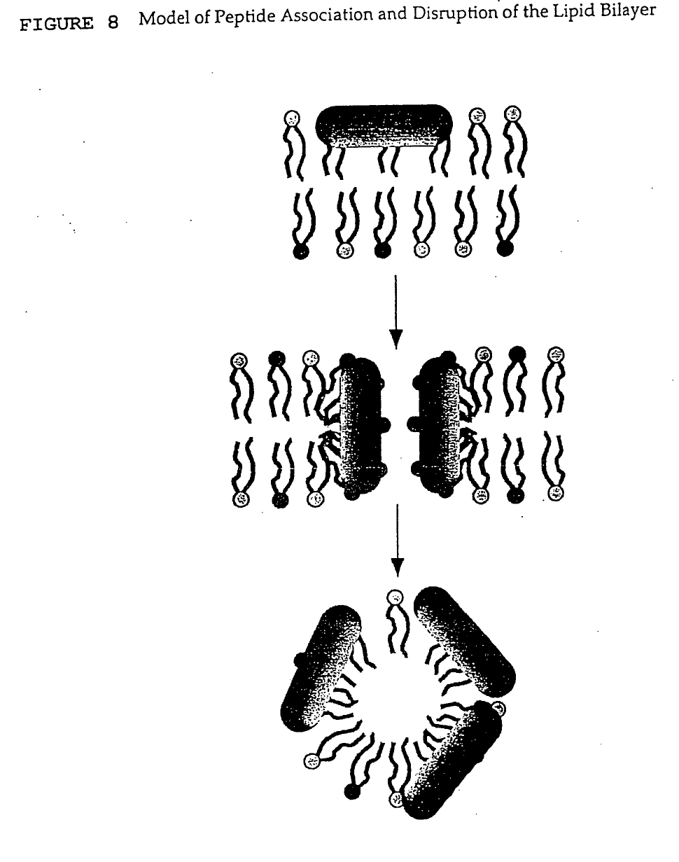

Fluorescence Emission in Tryptophan-Containing Analogs. W-KIAGKIA, W-KLAGLAK, and W-KIGAKI (see Table 1) were synthesized to assess the interaction of the peptides with LUV of varying lipid composition. Since each peptide contains a single tryptophan residue, fluorescence emission can be used to monitor binding and local polarity. The antimicrobial activity and secondary structure of these analogs were similar to those of the parent peptides (data not shown). In aqueous solution, the emission maxima for the three peptides were nearly identical (354-356 nm). The change in emission maximum to lower wavelength (blue shift) under different conditions is shown in FIG. 7. In 50% TFE, the emission maximum decreased by 4-5 nm for each peptide. Emission spectra in the presence of LUV were measured at lipid-to-peptide ratio of 20. The shift observed in the presence of POPC LUV (≦3 nm) was smaller than in 50% TFE for all peptides. The largest blue shifts were observed in the presence of POPG LUV. For W-KIAGKIA, the shifts in the presence of POPC/POPG were slightly larger than POPE/POPG at ratios above 1:1. This enhancement is much greater for W-KLAGLAK, where the blue shifts with POPC/POPG LUV are about twice as large as the corresponding POPE/POPG IS LUV at neutral-to-acidic ratios ≧2. The presence of either POPC or POPE decreased the magnitude of the blue shifts observed for W-KIGAKI to a much greater degree as compared to the other peptides; however, the shifts observed with POPE/POPG LUV were greater than those with POPC/POPG LUV at all ratios below 4.

The blue shifts observed for W-KIAGKIA in the presence of LUV containing E. coli lipids or ternary mixtures of POPC/POPG/DPG or POPE/POPG/DPG were nearly equal. For W-KLAGLAK, however, the shift observed in the presence of POPC/POPG/DPG was greater than with either E. coli lipids or POPE/POPG/DPG. Only W-KIGAKI showed larger blue shifts in the presence of E. coli lipids or POPE/POPG/DPG as compared to POPC/POPG/DPG.

DSC. An indication of the effects of membrane additives on the curvature properties of membranes may be assessed by their effect on the bilayer-to-hexagonal phase transition temperature (T_{H}) Only KLAGLAKx3 caused an appreciable increase in T_{H}. The change in T_{H} of DiPoPE as a function of peptide concentration is shown in Table 3.

Discussion

PGLa and the three model peptides, KIAGKIAx3, KLAGLAKx3, and KIGAKIx3, possess no defined secondary structure in solution, but adopt a conformation that appears to maximize amphipathic character upon interacting with lipid bilayers. Like PGLa, KIAGKIAx3 and KLAGLAKx3 can form an amphipathic α-helix at the bilayer surface. KIGAKIx3 was designed to mimic KIAGKIAx3 and KLAGLAKx3 in terms of net charge and hydrophobicity, but to form an amphipathic β-sheet instead of an α-helix. In 50% TFE, KIGAKIx3 is mainly helical, but when bound to LUV, the drive to form an amphipathic structure dominates and the resulting conformation is β-sheet as shown by CD and FTIR spectroscopy (FIGS. 2 and 3). A comparison of antimicrobial activity (Table 2) shows that KIAGKIAx3 and KIGAKIx3 are significantly more active than PGLa, with KLAGLAKs3 only slightly so. Notably, KIGAKIx3 is the least hemolytic of the three model peptides, and about the same as magainin 2 amide and PGLa.

PGLa, KIAGKIAx3, KLAGLAKx3, and particularly KIGAKI were not very effective at inducing leakage in PC LUV, but all were much more active with PG LUV. The binding of PGLa to membranes was shown recently to be dominated by electrostatic and not hydrophobic effects (47). Thus, increased binding probably accounts for the greater leakage rates observed in POPG vs. POPC LUV. At lower peptide levels, however, PGLa is more effective than the other peptides at inducing leakage in LUV containing POPG alone or POPC/POPG mixtures (FIG. 5). This contrasts with the antimicrobial activities (Table 2) that show PGLa as the least potent peptide. In LUV composed of E. coli polar lipids, however, the activity of PGLa is markedly reduced while that of KIGAKIx3 is enhanced compared to the other peptides (FIG. 4).

Since PE is the major uncharged polar lipid in E. coli plasma membranes, we examined the effect of replacing POPC by POPE. In LUV containing equimolar amounts of POPG and neutral lipid, only slight differences were observed. As the proportion of POPE in the LUV increased, however, the leakage rates more closely resembled those in E. coli LUV (FIG. 5). In a comparison of ternary mixtures of either POPE/POPG/DPG or POPC/POPG/DPG, the presence of PC greatly enhances the activity of PGLa and the other α-helical peptides, while reducing the activity of KIGAKIx3 (FIG. 6).

One may legitimately question whether the relatively high level of peptide necessary to induce leakage in LUV composed of E. coli polar lipid extract and other lipid mixtures with a high proportion of PE is relevant to the inhibitory effect upon bacterial growth. We can estimate the number of peptide molecules per bacterium in the MIC assay. The assay mixture contains 10⁵ bacteria in a volume of 0.2 mL. The lowest MIC value reported here is 8 μg/mL. With a molecular weight of ˜2,000, the amount of peptide in the assay (1.6 μg) translates to >4×10¹⁴ molecules. Thus, there are ˜4×10⁹ peptide molecules per bacterium even at the lowest MIC value. How does this relate to the number of lipid molecules in the plasma membrane? For a large bacterium of size 2×4 μm (i.e., even larger than the bacteria tested here), the surface area is ˜3×10⁷ nm². If the average surface area of a lipid molecule is estimated to be ˜0.7 nm², then the number of lipid molecules on the outer surface of the plasma membrane is ˜4×10⁷. Therefore, conservatively, there are about 100 peptide molecules for each lipid molecule on the exterior of the bacterial plasma membrane. For smaller bacteria or for higher MIC values, the number of peptides per lipid is proportionally higher.

This does not mean, however, that all of the peptides are bound to the plasma membrane. Many peptide molecules may be bound to lipopolysaccharide, peptidoglycan, teichoic acid, or other components of the cell envelope beyond the plasma membrane, while other peptides may remain free in solution.

The estimate does point out, however, that a real potential exists for a very large number of peptides to interact with the plasma membrane surface at antimicrobial concentrations. Further experiments will be necessary to determine the binding affinity and location of the peptides on intact bacteria.

What is the explanation for the observed differences between PC and PE in the leakage experiments? One obvious possibility is that the peptides bind differently to LUV containing PC or PE as the neutral lipid. We used tryptophan-containing analogs of the three model peptides to study their interactions with LUV. A blue shift in the maximum of the tryptophan emission band results from the decrease in polarity surrounding the indole side chain as the peptide binds to and penetrates the bilayer surface. Minimal and maximal blue shifts were observed in the presence of POPC and POPG LUV, respectively (FIG. 7). A comparison of LUV with either POPC/POPG or POPE/POPG reveals that W-KIAGKIA and W-KLAGLAK showed a greater interaction (i.e., a larger blue shift) with POPC/POPG while W-KIGAKI interacted more strongly with POPE/POPG. Thus, binding differences could contribute to the relative differences in leakage rates in LUV containing POPC vs. POPE. A second important factor affecting the activity of the peptides is, once bound, the precise nature of the interaction of the peptide with the membrane surface.

The curvature-modulating property (37) of KLAGLAKx3 differs from KIAGKIAx3 and KIGAKIx3 in that KLAGLAKx3 promotes more positive membrane curvature (Table 3). This property appears to have consequences for the lipid dependence of the lytic activity of magainin 2, which was shown to induce positive curvature to a slightly greater extent than KLAGLAKx3 (38). The curvature effects of these peptides can be rationalized in terms of their structure. Comparing the two α-helical model peptides, in KIAGKIAx3 the six lysine residues are clustered together, while in KLAGLAKx3 they are separated by three glycine residues in a helical wheel projection (FIG. 1). Lysines have a special role in the binding of peptides to bilayers because of the amphiphilic nature of their side chain (i.e., four hydrophobic methylene groups between the α-carbon atom and the side chain amino group) (59). In the case of KIAGKIAx3, the clustered lysine residues will allow the peptide to insert more deeply in the bilayer and thereby promote less positive curvature (60). In KLAGLAKx3, the two groups of lysine residues are at the interface between the hydrophobic and hydrophilic sides of the amphipathic helix as they are in Class A peptides (61), resulting in increased positive curvature. This difference in insertion might not be reflected in the fluorescent properties of the tryptophan-substituted analogs because the W residues should seek a position close to the interface, regardless of the depth of insertion of the peptide as a whole (62-64). Also, in W-KIAGKIA, the substitution is closer to the hydrophilic face as compared to W-KLAGLAK, where it is near the center of the hydrophobic face (FIG. 1). Since the β-sheet peptide, KIGAKIx3, does not shift T_{H} of DiPoPE, its lytic activity may not be dependent on the curvature properties of the bilayer surface.

The toroidal pore mechanism proposed by Matsuzaki (27) and Huang (65) for the antimicrobial action of magainin 2 is based upon the induction of sufficient positive curvature to create supramolecular pores. An alternative “carpet” model was proposed earlier by Shai and coworkers (39) which also relies upon changes in bilayer curvature to disrupt the membrane. Of the peptides examined in this work, only KLAGLAK resembles magainin 2 in its ability to generate positive curvature. Both KIAGKIAx3 and KIGAKIx3 have only a negligible effect on the T_{H} of DiPoPE. If the interactions with DiPoPE can be generalized to other lipids, then the membrane disruption caused by KIAGKIAx3 and KIGAKIx3 may well be different from magainin-like peptides.

We have demonstrated that KIGAKIx3, designed to adopt a highly amphipathic β-sheet, possesses a combination of equivalent antimicrobial activity and superior selectivity compared to the α-helical peptides in this study.

### EXAMPLE 2

In this study, we compared the structure and activity of:

| | | |
|---|---|---|
| | 1 5 10 15 20 | |
| KIAGKIAx3 | K I A G K I A K I A G K I A K I A G K I A-NH₂ | (SEQ ID NO:4) |
| KIGAKIx3 | K I G A K I K I G A K I K G A K I-NH₂ | (SEQ ID NO:6) |
| SH-KIGAKI | K I G A K S K H G A K I K I G A K I-NH₂ | (SEQ ID NO:25) |
| KL-14 | K L K L K L K W K L K L K L-NH₂ | (SEQ ID NO:26) |
| KL-18 | K L K L K L K W K L K L K L K L K L-NH₂ | (SEQ ID NO:27) |

KL-14 is a 14-residue KL repeat containing a tryptophan at position 8 in place of a leucine. KL-18 is the corresponding 18-residue peptide. Both of these peptides are more highly charged but much less in hydrophobic than our original β-sheet peptide, (KIGAKI)₃-NH₂ (SEQ ID NO:8). Both of these peptides should be highly amphipathic β-sheet peptides.

The conformation of the peptides in aqueous solution, in 50% TFE, and bound to lipid bilayers was determined by FTIR and CD spectroscopy. The peptides were compared for their ability to release the fluorescent dye, calcein, from LUVs composed of either PC, PG, 1:1 PC/PG, 2:1 PC/PG, 3:1 PC/PG, 4:1 PC/PG, 1:1 PE/PG, 2:1 PE/PG, 3:1 PE/PG, 4:1 PE/PG, or E. coli polar lipid extract. Interactions with the same set of LUVs were monitored by the tryptophan emission specturm of four of the peptides: KL-14, KL-18, and the analogs W₉-KIAGKIA and W₈-KIGAKI. These results then were compared to the antimicrobial activity of the peptides toward Escherichia coli, Staphylococcus aureus, and Pseudomonas aeruginosa in order to assess relationships between peptide structure and function.

The data is summarized in Table 4.

Results and Discussion

As shown previously, KIAGKIAx3 and KIGAKIx3 adopt alpha-helical and beta-sheet conformations, respectively, when bound to LUVs containing either pure PG or PG mixed with either PC or PE. KL-14 and KL-18 also adopt beta-sheet conformation under these conditions. SH-KIGAKI is predominantly beta-sheet in the presence of PG LUV, but not with either PC/PG or PE/PG LUVs.

KL-18 was most potent in inducing leakage from PC LUVs, followed by KL-14 and KIAGKIAx3. Both KIGAKI and SH-KIGAKI showed low lytic activity even at high peptide concentrations.

In PC/PG LUVs, the order of potency was KIAGKIAx3>KL-18>KL-14=KIGAKIx3>>SH-KIGAKI. The potency of KIAGKIAx3 was diminished substantially when PE replaced PC as the neutral lipid in LUVs. Only KIGAKIx3 was more active with PE/PG vs. PC/PG LUVs at all molar lipid ratios tested (1:1, 2:1, 3:1, and 4:1). SH-KIGAKI showed no ability to induce leakage in any LUVs containing PC/PG or PE/PG. In LUVs composed of E. coli polar lipids, the order of lytic potency was KL-1>KL-14>KIGAKIx3>>KIAGKIAx3)>SH-KIGAKI.

In experiments to estimate peptide binding to LUVs, W₈-KIGAKIx3 differed from WG-KIAGKIA, KL-14, and KL-18, in that it showed much larger blue shifts in the tryptophan emission maximum in PE-containing as opposed to PC-containing LUVs. The low affinity of KIGAKIx3 for PC-containing LUVs is consistent with the low lytic activity observed in these vesicles.

These antimicrobial activities of all peptides were assessed against E. coli, S. aureus, and P. aeruginosa. The potencies of KIAGKIAx3 and KIGAKIx3 were about equal. Both KL-14 and KL-18 showed higher MIC values (4-16 μg/mL) than KIAGKIAx3 or KIGAKIx3.

Thus, the antimicrobial activity of the KL peptides is lower than that of both KIAGKIAx3 and KIGAKIx3. The KL peptides also are much less selective than (KIGAKI)₃-NH₂.

We conclude that: (1) both amphipathic beta-sheet and alpha-helical peptides can possess comparable antimicrobial activity; (2) a relatively small reduction in amphipathic character (as shown by SH-KIGAKI) can result in a large loss of activity; (3) KL-14 and especially KL-18, which are both highly effective-at inducing leakage in LUVs from E. coli lipids, are less antimicrobial than KIAGKIA or KIGAKI (hydrophobicity may be an important factor here); and (4)KIGAKI is unique among these peptides since binding and leakage experiments demonstrate it to be highly selective for membranes containing PE as the neutral lipid instead of PC. KIGAKI therefore may have a higher therapeutic index than the other peptides since PC is a major-component in mammalian. plasma membranes. Since KL-14 and KL-18 are quite lytic toward PC LUVs, this selectivity clearly is not conferred by secondary structure alone.

### HYPOTHETICAL EXAMPLE 101

We intend to study two series of peptides of identical charge and hydrophobicity. Each model peptide will contain six lysine residues, and will be amidated at the carboxyl terminus. The first series will consist of five peptides, each containing a different hexamer sequence of the same set of amino acids repeated three times (18 residues). These peptides will possess no potential to form amphipathic α-helices, but they will have varying capacity to form amphipathic β-sheet structures. A second series will consist of six peptides, each containing a different heptamer sequence of the same set of amino acids repeated three times (21 residues). These peptides will have low amphipathic β-sheet potential, but will vary considerably in the prospect of forming amphipathic α-helices.

These two sets of peptides will enable us to examine a variety of structure/function relationships. Our experimental approach will be as follows:

1) Test the antimicrobial and hemolytic activity of each peptide.

2) Determine the secondary structure of the peptides in solution and bound to lipids.

3) Measure the ability of the peptides to induce leakage in LUV of varying lipid composition.

4) Measure the ability of the peptides to induce leakage in a strain of E. coli with constitutive β-galactosidase activity.

5) Determine how many peptide molecules must bind to the membrane in order to increase permeability in both LUV and bacteria.

101.1. Peptide Design and Synthesis

As described in the previous section, we have worked with two 21-residue peptides containing heptamer repeats and one 18-residue peptide containing a hexamer repeat. We propose here to create two complete peptide families. All of the peptides will be amidated at the carboxyl terminus. The first series will be comprised of 18-residue molecules made up of a trimeric hexamer repeat in which there are two lysines. One will be fixed at position 1 and the other will be located at either position 2, 3, 4, 5, or 6. The other amino acids in the hexamer repeat—I, G, A, I—will remain constant. The 18-residue family will consist of the following: 1,2-Hex=(KKIGAI)₃-NH₂; 1,3-Hex=(KIKGAI)₃-NH₂; 1,4-Hex=(KIGKAI)₃-NH₂; 1,5-Hex=(KIGAKI)₃-NH₂; and 1,6-Hex=(KIGAIK)₃-NH₂. Similarly, the second family will be comprised of 21-residue molecules made up of a trimeric heptamer repeat in which there are two lysines. One will be fixed at position 1 and the other will be located at either position 2, 3, 4, 5, 6, or 7.

The other amino acids in the heptamer repeat—L, A, G, L, A—will remain constant. The 21-residue family will consist of the following: 1,2-Hept=(KKLAGLA)₃-NH₂; 1,3-Hept=(KLKAGLA)₃-NH₂; 1,4-Hept=(KLAKGLA)₃-NH₂; 1,5-Hept=(KLAGKLA)₃-NH₂; 1,6-Hept=(KLAGLKA)₃-NH₂; and 1,7-Hept=(KLAGLAK)₃-NH₂.

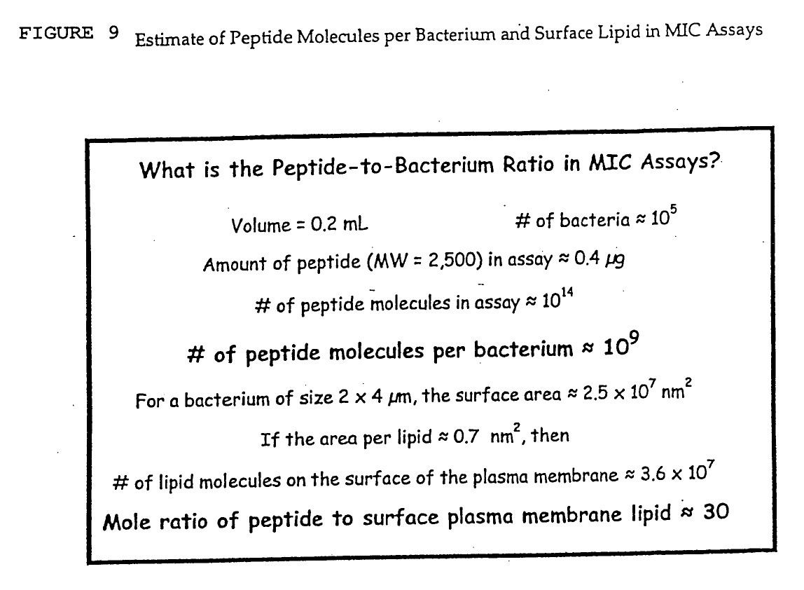

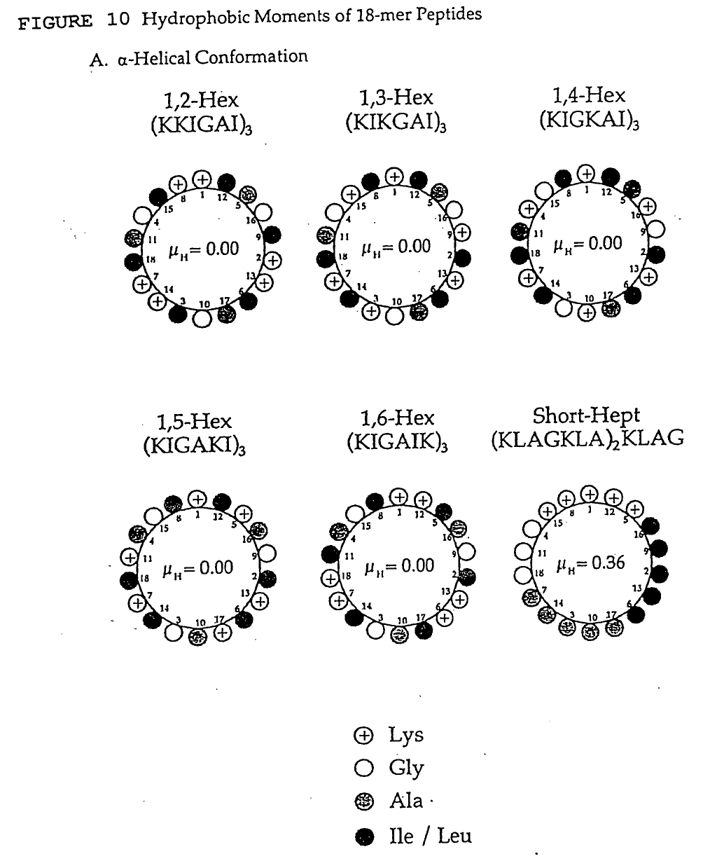

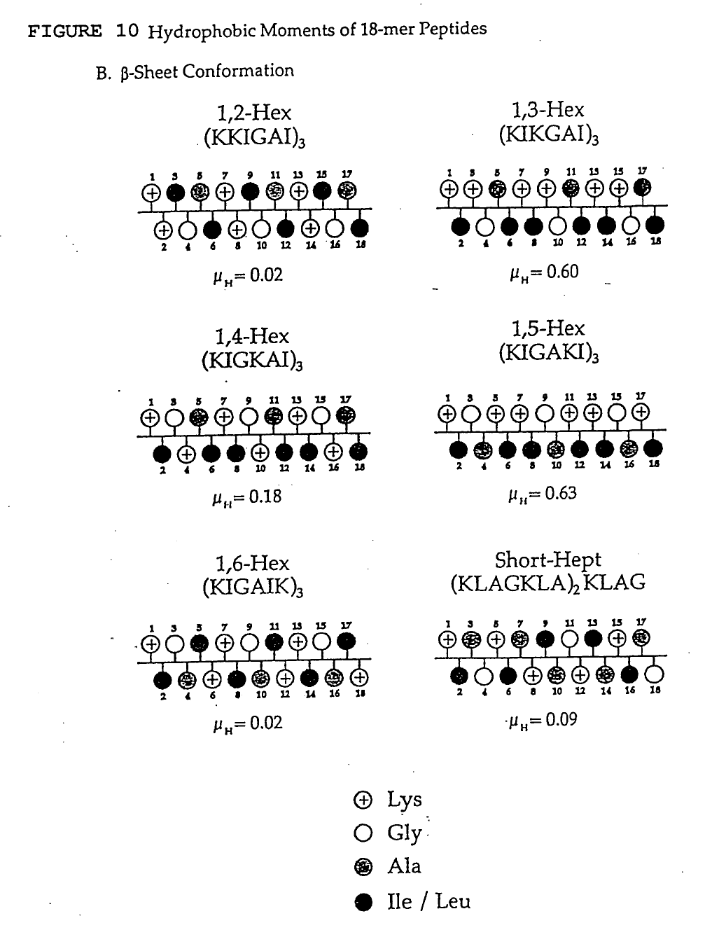

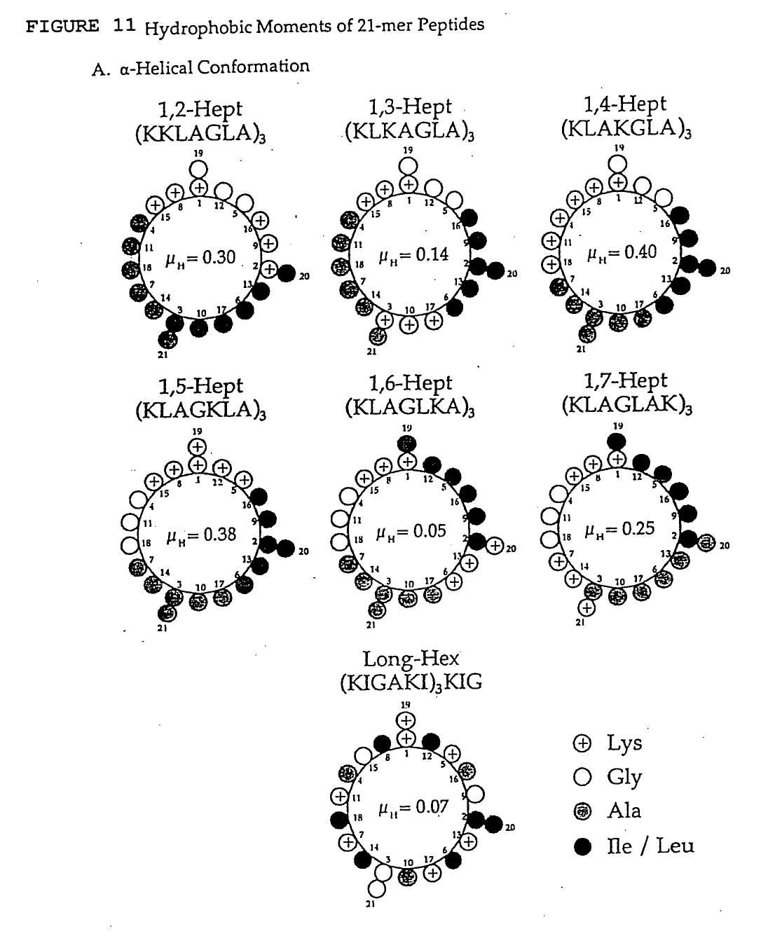

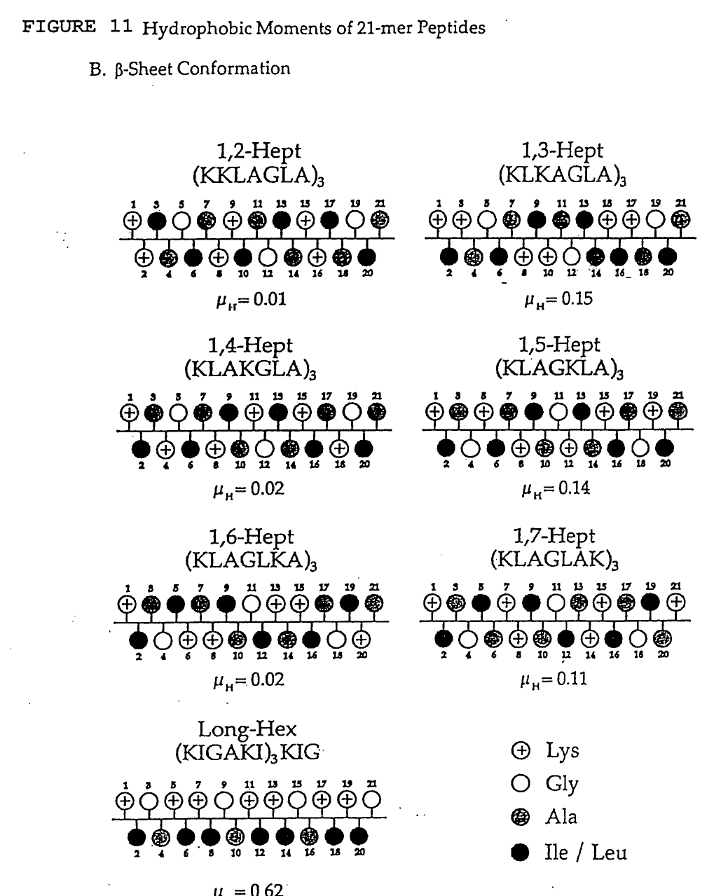

All of these peptides are matched both in terms of net charge (+7) and mean hydrophobicity (−0.06). The peptides will differ considerably, however, in their ability to form amphipathic α-helix or β-sheet structures. These two families will form the basis for comparisons in relating conformation and amphipathicity to activity. In addition, two more peptides will be made—Long-Hex (a 21-residue version of 1,5-Hex)=(KIGAKI)₃KIG-NH₂ and Short-Hept (an 18-residue version of 1,5-Hept)=(KLAGKLA)₂KLAG-NH₂. Long-Hex and Short-Hept will have a net charge of +8 and +6, respectively, while their mean hydrophobicities will be the same as the other peptides. These peptides should provide some insight into the effects of length and charge. The amphipathic potential as both α-helices and β-sheets of the 18-residue and 21-residue families are shown in FIGS. 8 and 9, respectively.

101.2. Antimicrobial and Hemolytic Activity

Antimicrobial susceptibility testing against Escherichia coli (ATCC 25922), Staphylococcus aureus (ATCC 29213), Pseudomonas aeruginosa (ATCC 27853), and Candida albicans (ATCC 14053) is performed using a modification of the National Committee for Clinical Laboratory Standards microdilution broth assay. Mueller-Hinton broth (BBL) is used for diluting the peptide stock solution and for diluting the bacterial inoculum. The inoculum is prepared from midlogarithmic phase cultures at an approximate concentration of 10⁶ CFU/mL. Microtiter plate wells receive aliquots of 0.1 mL each of the inoculum and peptide dilution. The final concentration of the peptide solution ranges from 0.25 to 256 μg/mL in 2-fold dilutions. The microtiter plates are incubated overnight at 37° C. Minimum inhibitory concentration (MIC) is defined as the lowest concentration of peptide that completely inhibits growth of the organism.

Hemolytic activity is determined by adding a defined concentration of peptides to a 5% suspension of freshly drawn human erythrocytes, which had been washed twice in phosphate buffered saline. After incubation at 37° C. for 30 minutes, the suspension is centrifuged at 10,000×g for 10 minutes and the absorbance at 400 nm is measured. Complete hemolysis is determined by adding 0.2% Triton X-100 in place of the peptides.

101.3. Peptide Conformation

The conformation of the peptides will be assessed by FTIR and CD spectroscopy. The data presented in the previous section demonstrates the utility of these methods for determining peptide conformation both in solution and when bound to lipids.

101.3.1. FTIR Spectroscopy. Mixtures of lipid (4 μmoles) and peptide (varied to achieve the desired peptide-to-lipid ratio) are codissolved in 2:1 CHCl₃/CH₃OH. The solvent is evaporated and the sample is placed under high vacuum for 3 hours. The mixture is hydrated with D₂O buffer (20 mM PIPES, 1 mM EGTA, pD 7.0) and incubated at 50° C. for 1 hour. The sample is isolated by centrifugation and placed between CaF₂ windows separated by a 25 μm Teflon spacer. Infrared spectra are collected using a Mattson Polaris FTIR spectrometer with a HgCdTe detector. A total of 256 interferograms are co-added and Fourier transformed with triangular apodization to generate absorbance spectra with 2 cm⁻¹ resolution and data points encoded every 1 cm⁻¹, with a signal-to-noise ratio of better than 500. Evaluation of secondary structure from the amide I′ band between 1600 and 1700 cm⁻¹ is performed as described in the preliminary results section. When necessary, Fourier deconvolution and curve fitting are used to measure the subcomponents of the amide I′ band.²² All of the peptides will be tested in lipid systems consisting of pure PG as well as PC/PG, PE/PG, and E.coli lipid mixtures at peptide-to-lipid ratios ranging from 1:20 to 1:2.

101.3.2. CD Spectroscopy. CD spectra are measured using a Jasco J-715 spectropolarimeter. Spectra are recorded from 250-190 nm at a sensitivity of 100 mdeg, resolution of 0.1 nm, response of 8 seconds, bandwidth of 1.0 nm, and scan speed of 20 nm/min, with a single accumulation. For the lowest peptide concentrations, a response of 16 seconds, scan speed of 10 nm/min, and a total of 5 accumulations are used. The buffer contains 10 mM potassium phosphate, 150 mM KCl, 1 mM EDTA, pH 7.0. The peptide concentration in buffer and TFE/buffer mixtures is 20 μM. LUV are prepared from aqueous dispersions of the appropriate lipid systems (containing POPG, POPC/POPG, POPE/POPG, or E. coli lipids) at a concentration of ˜1 mg/mL in phosphate buffer. Following 5 freeze/thaw cycles, the mixture is extruded 10 times through a 0.1-μm-pore polycarbonate membrane in an Avanti mini-extruder apparatus, resulting in ˜100-nm-diameter LUV. The lipid concentration is determined by a phosphorus assay.³⁹ The lipid concentration in the cuvet is kept constant at 100 μM. The peptide concentration is varied from 1 μM to 50 μM to achieve peptide-to-lipid ratios ranging from 1:100 to 1:2. The helical content can be estimated using the method of Luo and Baldwin.⁴⁰

101.4. Peptide-Induced Calcein Leakage from LUV

The ability of the peptides to release calcein (MW 623) from LUV of varying lipid composition will be compared. LUV are prepared as above, except that the buffer consists of 50 mM HEPES, 100 mM NaCl, 0.3 mM EDTA, 80 mM calcein, pH 7.4. Calcein-loaded vesicles are separated from free calcein by size-exclusion chromatography using a Sephadex G-50 column. Calcein leakage is monitored by measuring the time-dependent increase in fluorescence of calcein (excitation=490 nm, emission=520 nm). LUV containing 8 nmoles of lipid are added to 1.5 mL of buffer in a stirred cuvet at 25° C. An aliquot of peptide is added to achieve the desired peptide-to-lipid ratio (1:256 to 1:2). Complete leakage is determined by the addition of 20 pL of 10% Triton X-100.

101.5. Peptide-Induced Calcein Leakage in E. coli

We have obtained a strain of E. coli (ML-35) from Dr. Renato Gennaro (University of Trieste, Italy). This strain is lactose permease-deficient and constitutive for cytoplasmic β-galactosidase. Using o-nitrophenyl-β-D-galactopyranoside (ONPG) as a substrate, it is possible to evaluate peptide-induced permeabilization of the plasma membrane. In the absence of peptide, ONPG is impermeable to the cell. The increase in permeability is monitored colorimetrically by incubated 10⁶ CFU of bacteria in 10 mM sodium phosphate, 100 mM NaCl, 1.5 mM ONPG, pH 7.5, in a cuvet at 37° C. Following the addition of peptide, the entry of ONPG into the cell is monitored by the increase in absorbance at 405 nm due to the production of o-nitrophenol.⁴¹ We will determine the concentration of each peptide necessary to induce leakage and also examine the effect of changing the number of bacteria in the assay. This should provide some clue as to the number of peptides per bacteria that are necessary to induce leakage.

101.6. Peptide Binding to LUV and Bacteria

We will attempt to measure peptide binding to bacteria in three different ways:

101.6.1. Physical separation of peptides from LUV or bacteria. We have used microfiltration tubes in preparing samples for FTIR spectroscopy. These tubes, with a 0.4 μm nylon filter, can separate particulates (e.g., LUV) from the filtrate. By simply measuring the amount of peptide in the filtrate, it may be possible to determine how much peptide is bound. There are potential problems with this method, however. Particularly at higher peptide levels, if the LUV are breaking down, the filter may not be able to retain all of the lipid. An equilibrium dialysis using a low molecular weight cutoff membrane that allows individual peptides to pass through but not supramolecular complexes may be an alternative to microfiltration tubes. A similar approach will be attempted for bacteria by incubating the cells with peptide and separating the bacteria from the solution by centrifugation. The amount peptide remaining in the supernatant will be identified using either HPLC or a peptide-specific antibody.

101.6.2. Tryptophan emission of fluorescent peptide analogues. We have made the following fluorescent analogues of (KIAGKIA)₃-NH₂, (KLAGLAK)₃-NH₂, and (KIGAKI)₃-NH₂.

| | | |
|---|---|---|
| 1 | KIAGKIAKWAGKIAKTAGKIA- NH₂ | (SEQ ID NO:5) |
| 2 | KLAGLAKKWAGLAKKLAGLAK- NH₂ | (SEQ ID NO:7) |
| 3 | KIGAKIKWGAKIKIGAKI- NH₂ | (SEQ ID NO:9) |

In each case, the tryptophan replacement caused no appreciable change in antimicrobial or hemolytic activity. The binding of these peptides to lipid bilayers can be detected by a significant blue shift in the tryptophan emission maximum. In water, the emission maximum is near 356 nm. In the presence of LUV, if the peptide is completely. bound, the emission maximum. shifts to near 330 nm as the tryptophan associates with the lipid bilayer.

The antimicrobial activity and secondary structure of these analogs were similar to those of the parent peptides. The change in emission maximum to lower wavelength (blue shift) under different conditions is shown in FIG. 7. In 50% TFE, the emission maximum decreased by 4-5 nm for each peptide. Emission spectra in the presence of LUVs were measured at lipid-to-peptide ration of 20. The shift observed in the presenced of POPC LUVs (≦3 nm) was smaller than in 50% TFE for all peptides. The largest blue shifts were observed in the presence of POPG LUVs. For W-KIAGKIA, the shifts in the presence of POPC/POPG were slightly larger than POPE/POPG at ratios above 1:1. This enhancement is much greater for W-KLAGLAK, where the blue shifts with POPC/POPG LUVs are about twice as large as the corresponding POPE/POPG LUVs at neutral-to-acidic ratios 22. The presence of either POPC or POPE decreased the magnitude of the blue shifts observed for W-KIGAKI to a much greater degree as compared to the other peptides; however, the shifts observed with POPE/POPG LUVs were greater than those with POPC/POPG LUVs at all ratios below 4. The blue shifts observed for W-KIAGKIA in the presence of LUVs containing E. coli lipids or ternary mixtures of POPC/POPG/DPG or POPE/POPG/DPG were nearly equal. For W-KLAGLAK, however, the shift observed in the presence of POPC/POPG/DPG was greater than with either E. coli lipids or POPE/POPG/DPG. Only W-KIGAKI showed larger blue shifts in the presence of E. coli lipids or POPE/POPG/DPG as compared to POPC/POPG/DPG. Similar binding experiments may be performed with the heptamer and hexamer peptide families.

101.6.3. Varying the number of bacteria in MIC assays. As described above, the number of bacteria used in MIC assays is 10⁵. We can gauge the number of peptide molecules necessary to cause killing by altering the number of bacteria in the assay and observing the effect on the MIC values. For instance, if thp MIC value increases in proportion to the number of peptide molecules remain available in solution to bind to and kill the cells, given the increased target load. On the other-hand, if no difference is observed in the MIC value as the number of bacteria in the assay increases, then the concentration of peptides in solution must remain sufficiently high to accommodate the increased number of cells. If this is the case, then the binding affinity at the MIC level rather than the absolute amount of peptide in the assay is the limiting factor.

The results of these binding studies should provide some insight into the actual number of peptide.molecules required to induce leakage in LUV and bacterial cells.

### EXAMPLE 102

We have synthesized the hexalmer and heptamer peptide families described in hypothetical example 101. These peptides were made with a single tryptophan residue to permit fluorescence experiments. The sequences are:

| | |
|---|---|
| Hexamer Family | |
| 1,2-Hex | |
| K K I G A I K K W G A J K K I G A I-NH₂ | (SEQ ID NO:25) |
| 1 5 10 15 | |
| 1,3-Hex | |
| K I K G A I K W K G A I K I K G A I-NH₂ | (SEQ ID NO:26) |
| 1,4-Hex | |
| K I G K A I K W G K A I K I G K A I-NH₂ | (SEQ ID NO:27) |
| 1,5-Hex | |
| K I G A K I K W G A K I K I G A K I-NH₂ | (SEQ ID NO:29) |
| 1,6-Hex | |
| K I G A I K K W G A I K K I G A I K-NH₂ | (SEQ ID NO:30) |
| Heptamer Family | |
| 1,2-Hept | |
| K K L A G L A K K W A G L A K K L A G L A-NH₂ | (SEQ ID NO:31) |
| 1 5 10 15 20 | |
| 1,3-Hept | |
| K L K A G L A K W K A G L A K L K A G L A-NH₂ | (SEQ ID NO:32) |
| 1,4-Hept | |
| K L A K G L A K W A K G L A K L A K G L A-NH₂ | (SEQ ID NO:33) |
| 1,5-Hept | |
| K L A G K L A K W A G K L A K L A G K L A-NH₂ | (SEQ ID NO:34) |
| 1,6-Hept | |
| K L A G L K A K W A G L K A K L A G L K A-NH₂ | (SEQ ID NO:35) |
| 1,7-Hept | |
| K L A G L A K K W A G L A K K L A G L A K-NH₂ | (SEQ ID NO: 36) |

The physical properties and antimicrobial activities of these peptides are shown in Table 102.

None of the peptides in the hexamer family has the capacity to form an amphipathic α-helix. Only those peptides that can form a highly amphipathic β-sheet (i.e., 1,3-Hex and 1,5-Hex) have appreciable antimicrobial activity. For the heptamer family, the peptides that can adopt an amphipathic α-helix (1,2-Hept, 1,4-Hept, 1,5-Hept, and 1,7-Hept) all have relatively low MIC values. 1,6-Hept, which has no amphipathic potential as either an α-helix or β-sheet, is devoid of antimicrobial activity, while 1,3-Hept, with some amphipathic potential as both an α-helix and β-sheet, is slightly active.

### EXAMPLE 103

### N-Terminal Modified Peptides

We have made five derivatives of our β-sheet peptides with an octanoyl [CH₃—(CH₂)₆—(C═O)—] group added to the amino terminus. Four are derivatives of KIGAKI (1-3 repeats) and one is a derivative of a KL repeat. All of these peptides contain a single tryptophan for fluorescence studies, but this could be replaced by I or L in any final product. The sequences are:

| | |
|---|---|
| KIGAKI | |
| K I G A K I K I G A K I K I | (SEQ ID NO:8) |
| G A K I-NH₂ | |
| Oct-KIGAKI-18A | |
| Oct- K I G A K I K W G A K I K I | (SEQ ID NO:9) |
| G A K I-NH₂ | |
| Oct-KIGAKI-18B | |
| Oct- K I G A K I K I G A K I K W | (SEQ ID NO:37) |
| G A K I-NH₂ | |
| Oct-KIGAKI-12 | |
| Oct- K I G A K I K W G A K I-NH₂ | (SEQ ID NO:38) |
| Oct-KIGAKI-6 | |
| Oct- K W G A K I-NH₂ | (SEQ ID NO:39) |
| Oct-Beta-11 | |
| Oct- K L K W K L K L K L K-NH₂ | (SEQ ID NO:40) |
| KL-14 | |
| K L K L K L K W K L K L K | (SEQ ID NO:26) |
| L-NH₂ | |
| KL-18 | |
| K L K L K L K W K L K L K | (SEQ ID NO:27) |
| L K L K L-NH₂ | |

The antimicrobial activity of the octanoylated peptides in comparison to related peptides is set forth in Table 103.

Adding an octanoyl group to the 18-residue KIGAKI did not appear to increase potency to a great extent. We have not yet measured the hemolytic activity of these octanoylated peptides, nor have.we yet tested a 12-residue octanoylated KIGAKI. A 6-residue octanoylated KIGAKI had no activity. Oct-Beta-11 is a shortened octanoylated version of KL-14 and KL-18. This peptide appears to have reasonable antimicrobial activity, but we have not measured its hemolytic activity and have not tested whether it possesses the desired selectivity between bacterial membrane lipids and mammalian membrane lipids.

### EXAMPLE 104

### Application of Combinatorial Chemistry to β-Sheet Framework

We have shown that our original peptide (KIGAKI)3-NH2 (1,5-Hex) and 1,3-hex have antimicrobial-activity. In an effort to increase potency and maintain selectivity, we could employ a combinatorial approach to scarch for derivatives. This is a common method that is used in many laboratories (see, for example, Houghten, R. A.; Pinilla, C.; Blondelle, S. E.; Appel, J. R.; Dooley, C. T., and Cuervo, J. H. Generation and use of synthetic peptide combinatorial libraries for basic research and drug discovery. Nature, 1991, 354:84-86.

Using this method, we could retain the amphipathic β-sheet architecture (controlling length and charge) while searching for favorable amino acid replacements. For example, we could try the following:

| K X G X K X K X G X K X K X | (SEQ ID NO. 42) |
|---|---|
| 1 5 10 | |
| G X K X-NH₂ | |
| 15 | |

We could screen a set of 15 hydrophobic amino acids (these could be natural and/or unnatural) at the positions marked X in the sequence. Starting at position 2, we would make 15 separate libraries, each with a unique amino acid at position 2, and an equimolar mixture of the 15 amino acids at the other X positions. These 15 libraries (each containing millions of different peptides) would then be screened for antimicrobial and hemolytic activity. The library or libraries giving the the best results identify candidate amino acids for position 2. This is then repeated by making 15 libraries each for positions 4, 6, 8, 10, 12, 14, 16 and 18. After the best amino acids were identified at each position (a process called positional scanning), then individual peptides would be made to test whether they are superior to our original compounds.

It is noted that this specification may, from time to time, refer to “KIGAKI”, “KIAGKIA”, etc., when the respective trimers (KIGAKI)3, (KIAGKIA)3, etc., are intended. The nomenclature “KIGAKIx3”, “KIAGKIAx3”, etc. is also used.

Literature Cited

1. Nicas, T. I., M. L. Zeckel, and D. K. Braun. 1997. Beyond vancomycin: New therapies to meet the challenge of glycopeptide resistance. Trends Microbiol. 5:240-249.

2. Zasloff, M. 1992. Antibiotic peptides as mediators of innate immunity. Curr.Opin.Immunol. 4:3-7.

3. Wade, D., A. Boman, B. Wahlin, C. M. Drain, D. Andreu, H. G. Boman, and R. B. Merrifield. 1990. All-D amino acid-containing channel-forming antibiotic peptides. Proc.Natl.Acad.Sci.U.S.A. 87:4761-4765.

4. Zasloff, M. 1987. Magainins, a class of antimicrobial peptides from Xenopus skin: isolation, characterization of two active forms, and partial cDNA sequence of a precursor. Proc.Natl.Acad.Sci.USA. 84:5449-5453.

5. Zasloff, M., B. Martin, and H. C. Chen. 1988. Antimicrobial activity of synthetic magainin peptides and several analogues. Proc.Natl.Acad.Sci.USA. 85:910-913.

6. Hoffmann, W., K. Richter, and G. Kreil. 1983. A novel peptide designated PYLa and its precursor as predicted from cloned mRNA of Xenopus laevis skin. EMBO J. 2:711-714.

7. Levy, S. B. 1992. The Antibiotic Paradox: How Miracle Drugs Are Destroying The Miracle. Plenum Press, New York.

8. Anderson, R. M. 1999. The pandemic of antibiotic resistance [news]. Nat. Med. 5:147-149.

9. Moellering, R. C., Jr. 1998. Antibiotic resistance: lessons for the future. Clin. Infect. Dis. 27 (Suppl. 1):S135-S142.

10. Walsh, C. T., S. L. Fisher, I. S. Park, M. Prahalad, and Z. Wu. 1996. Bacterial resistance to vancomycin: five genes and one missing hydrogen bond tell the story. Chem. Biol. 3:21-28.

11. Maloy, W. L. and U. P. Kari. 1995. Structure-activity studies on magainins and other host defense peptides. Biopolymers 37:105-122.

12. Jacob, L. S., and M. Zasloff. 1994. Potential therapeutic applications of magainins and other antimicrobial agents of animal origin. In Antimicrobial Pestides. John Wiley & Sons,^{—}Chichester. 197-223.

13. Chen, H.-C., J. H. Brown, J. L. Morell, and C. M. Huang. 1988. Synthetic magainin analogues with improved antimicrobial activity. FEBS Lett. 236:462-466.

14. Eisenberg, D. 1984. Three-dimensional structure of membrane and surface proteins. Annu.Rev.Biochem. 53:595-623.

15. Cuervo, J. H., B. Rodriguez, and R. A. Houghten. 1990. Synthesis and antimicrobial activity of magainin alanine substitution analogs. In Peptides. J. E. Rivier and G. R. Marshall, editors. ESCOM, Leiden. 124-126.

16. Rana, F. R., C. M. Sultany, and J. Blazyk. 1990. Interactions between Salmonella typhimurium lipopolysaccharide and the antimicrobial peptide, magainin 2 amide. FEBS Lett. 261:464-467.

17. Rana, F. R. and J. Blazyk. 1991. Interactions between the antimicrobial peptide, magainin 2, and Salmonella typhimurium lipopolysaccharides. FEBS Lett. 293:11-15.

18. Rana, F. R., E. A. Macias, C. M. Sultany, M. C. Modrzakowski, and J. Blazyk. 1991. Interactions between magainin 2 and Salmonella typhimurium outer membranes: effect of lipopolysaccharide structure. Biochemistry 30:5858-5866.

19. Matsuzaki, K., M. Harada, S. Funakoshi, N.-Fujii, and K. Miyajima. 1991. Physicochemical determinants for the interactions of magainins 1 and 2 with acidic lipid bilayers. Biochim.Biophys.Acta 1063:162-170.

20. Matsuzaki, K. 1998. Magainins as paradigm for the mode of action of pore forming polypeptides. Biochim.Biophys.Acta Rev.Biomembr. 1376:391-400.

21. Bechinger, B., M. Zasloff, and S. J. Opella. 1993. Structure and orientation of the antibiotic peptide magainin in membranes by solid-state nuclear magnetic resonance spectroscopy. Protein.Science 2:2077-2084.

22. Hirsh, D. J., J. Hammer, W. L. Maloy, J. Blazyk, and J. Schaefer. 1996. Secondary structure and location of a magainin analogue in synthetic phospholipid bilayers. Biochemistry 35:12733-12741.

23. Matsuzaki, K., O. Murase, H. Tokuda, S. Funakoshi, N. Fujii, and K. Miyajima. 1994. Orientational and aggregational states of magainin 2 in phospholipid bilayers. Biochemistry 33:3342-3349.

24. Ludtke, S. J., K. He, Y. Wu, and H. W. Huang. 1994. Cooperative membrane insertion of magainin correlated with its cytolytic activity. Biochim.Biophys.Acta Bio-Membr. 1190:181-184.

25. Duclohier, H., G. Molle, and G. Spach. 1989. Antimicrobial peptide magainin I from Xenopus skin forms anion-permeable channels in planar lipid bilayers. Biophys.J. 56:1017-1021.

26. Cruciani, R. A., J. L. Barker, S. R. Durell, G. Raghunathan, H. R. Guy, M. Zasloff, and E. F. Stanley. 1992. Magainin 2, a natural antibiotic from frog skin, forms ion channels in lipid bilayer membranes. Eur.J.Pharmacol.Mol.Pharmacol. 226:287-296.

27. Matsuzaki, K., O. Murase, N. Fujii, and K. Miyajima. 1996. An antimicrobial peptide, magainin 2, induced rapid flip-flop of phospholipids coupled with pore formation and peptide translocation. Biochemistry 35:11361-11368.

28. Ludtke, S. J., K. He, W. T. Heller, T. A. Harroun, L. Yang, and H. W. Huang. 1996. Membrane pores induced by magainin. Biochemistry 35:13723-13728.

29. Wieprecht, T., M. Dathe, M. Schümann, E. Krause, M. Beyermann, and M. Bienert. 1996. Conformational and functional study of magainin 2 in model membrane environments using the new approach of systematic double-D-amino acid replacement. Biochemistry 35:10844-10853.

30. Macias, E. A., F. Rana, J. Blazyk, and M. C. Modrzakowski. 1990. Bactericidal activity of magainin 2: use of lipopolysaccharide mutants. Can.J.Microbiol. 36:582-584.

31. Rana, F. R., C. M. Sultany, and J. Blazyk. 1991. Determination of the lipid composition of Salmonella typhimurium outer membranes by ³¹P NMR. J.Microbiol.Methods 14:41-51.

32. Gullion, T. and J. Schaefer. 1989. Rotational-echo double-resonance NMR. J.Magn.Reson. 81:196-200.

33. Krimm, S. and J. Bandekar. 1986. Vibrational spectroscopy and conformation of peptides, polypeptides, and proteins. Adv.Protein.Chem. 38:181-364.

34. Jackson, M., H. H. Mantsch, and J. H. Spencer. 1992. Conformation of magainin-2 and related peptides in aqueous solution and membrane environments probed by Fourier transform infrared spectroscopy. Biochemistry 31:7289-7293.

35. Williams, R. W., R. Starman, K. M. Taylor, K. Gable, T. Beeler, M. Zasloff, and D. Covell. 1990. Raman spectroscopy of synthetic antimicrobial frog peptides magainin 2a and PGLa. Biochemistry 29:4490-4496.

36. Blazyk, J., J. Hammer, A. E. Kearns, U. P. Kari, and W. L. Maloy. 1999. Conformation and activity of antimicrobial peptides related to PGLa. In Peptides: Frontiers of Peptide Science, J. P. Tam and P. T. P. Kaumaya, editors. Kluwer Academic Publishers, Dordrecht, The Netherlands. 385-386.

37. Epand, R. M. 1998. Lipid polymorphism and protein-lipid interactions. Biochim.Biophys.Acta Rev.Biomembr. 1376:353-368.

38. Matsuzaki, K., K. Sugishita, N. Ishibe, M. Ueha, S. Nakata, K. Miyajima, and R.M. Epand. 1998. Relationship of membrane curvature to the formation of pores by magainin 2. Biochemistry 37:11856-11863.

39. Chen, P. S., T. Y. Toribara, and H. Warner. 1956. Microdetermination of phosphorus. Anal.Chem. 28:1756-1758.

40. Luo, P. Z. and R. L. Baldwin. 1997. Mechanism of helix induction by trifluoroethanol: A framework for extrapolating the helix-forming properties of peptides from trifluoroethanol/water mixtures back to water. Biochemistry 36:8413-8421.

41. Skerlavaj, B., D. Romeo, and R. Gennaro. 1990. Rapid membrane permeabilization and inhibition of vital functions of gram-negative bacteria by bactenecins. Infection.&.Immunity. 58:3724-3730.

42. Zasloff, M. (1987) Proc. Natl. Acad. Sci. USA 84, 5449-5453.

43. Hoffmann, W., Richter, K., and Kreil, G. (1983) EMBO J. 2, 711-4.

44. Soravia, E., Martini, G., and Zasloff, M. (1988) FEBS Lett. 228, 337-340.

45. Bechinger, B., Zasloff, M., and Opella, S. J. (1993) Protein Sci. 2, 2077-2084.

46. Bechinger, B., Zasloff, M., and Opella, S. J. (1998) Biophys. J. 74, 981-987.

47. Wieprecht, T., Apostolov, O., Beyermann, M., and Seelig, J. (2000) Biochemistry 39, 442-452.

48. Epand, R. M. and Vogel, H. J. (1999) Biochim. Biophys. Acta 1462, 11-28.

49. Sitaram, N. and Nagaraj, R. (1999) Biochim. Biophys. Acta 1462, 29-54.

50. Osterman, D. G. and Kaiser, E. T. (1985) J. Cell Biochem. 29, 57-72.

51. Krimm, S. and Bandekar, J. (1986) Adv. Protein Chem. 38, 181-364.

52. Blondelle, S. E. and Houghten, R. A. (1992) Biochemistry 31, 12688-12694.

53. Ono, S., Lee, S., Mihara, H., Aoyagi, H., Kato, T., and Yamasaki, N. (1990) Biochim. Biophys. Acta 1022, 237-44.

54. Oh, J. E., Hong, S. Y., and Lee, K. H. (1999) J. Pept. Res. 53, 41-46.

55. Castano, S., Desbat, B., and Dufourcq, J. (2000) Biochim. Biophys. Acta 1463, 65-80.

56. Oren, Z., Hong, J., and Shai, Y. (1997) J. Biol. Chem. 272, 14643-14649.

57. Hong, J., Oren, Z., and Shai, Y. (1999) Biochemistry 38, 16963-16973.

58. Oren, Z., Hong, J., and Shai, Y. (1999) Eur. J. Biochem. 259, 360-369.

59. Mishra, V. K., Palgunachari, M. N., Segrest, J. P., and Anantharamaiah, G. M. (1994) J. Biol. Chem. 269, 7185-91.

60. Tytler, E. M., Segrest, J. P., Epand, R. M., Nie, S. Q., Epand, R. F., Mishra, V. K., Venkatachalapathi, Y. V., and Anantharamaiah, G. M. (1993) J. Biol. Chem. 268, 22112-22118.

61. Segrest, J. P., De Loof, H., Dohlman, J. G., Brouillette, C. G., and Anantharamaiah, G. M. (1990) Proteins 8, 103-17.

62. White, S. H., Wimley, W. C., Ladokhin, A. S., and Hristova, K. (1998) Methods Enzymol. 295, 62-87.

63. Yau, W. M., Wimley, W. C., Gawrisch, K., and White, S. H. (1998) Biochemistry 37, 14713-8.

64. Wimley, W. C. and White, S. H. (1996) Nat. Struct. Biol. 3, 842-8.

65. Ludtke, S. J., He, K., Heller, W. T., Harroun, T. A., Yang, L., and Huang, H. W. (1996) Biochemistry 35, 13723-13728.

66. Pouny, Y. and Shai, Y. (1992) Biochemistry 31, 9482-90.

All references cited herein, including journal articles or abstracts, abandoned or pending (whether or not published) U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass or subrange contained therein, as well as a separate description of each individual member or value in said class or range.

Where a set of preferred embodiments are recited for particular elements of the invention, any combination of a first set of preferred embodiments for a first element, with a second set of preferred elements for a second element, shall also be considered a preferred embodiment, and so forth for higher combinations including additional elements.

TABLE 1
Amino Acid Sequences of Antimicrobial Peptides

| | | |
|---|---|---|
| | 1 5 10 15 20 | |
| Magainin2-NH2 | G I G K F L H S A K K F G K A F V G E I M N S -NH₂ | (SEQ ID NO:1) |
| PGLa | G M A S K A G A I A G K I A K V A L K A L -NH₂ | (SEQ ID NO:2) |
| G1K, G8K-PGLa | K L A S K A G K I A G K I A K V A L K A L -NH₂ | (SEQ ID NO:3) |
| KIAGKIAx3 | K I A G K I A K I A G K I A K I A G K I A -NH₂ | (SEQ ID NO:4) |
| W-KIAGKIA | K I A G K I A K W A G K I A K I A G K I A -NH₂ | (SEQ ID NO:5) |
| KLAGLAKx3 | K L A G L A K K L A G L A K K L A G L A K -NH₂ | (SEQ ID NO:6) |
| W-KLAGLAK | K L A G L A K K W A G L A K K L A G L A K -NH₂ | (SEQ ID NO:7) |
| KIGAKIx3 | K I G A K I K I G A K I K I G A K I -NH₂ | (SEQ ID NO:8) |
| W-KIGAKI | K I G A K I K W G A K I K I G A K I -NH₂ | (SEQ ID NO:9) |

TABLE 2

| | Minimum Inhibitory Concentration (μg/mL)^{b} | | | | % Hemolysis |
|---|---|---|---|---|---|
| Peptide^{a} | E. coli | S. aureus | P. aeruginosa | C. albicans | (500 μg/mL) |
| Magainin 2-NH₂ | 64 | 256 | 256 | >256 | 2 |
| PGLa | 32 | 64 | 128 | 128 | 1 |
| (KIAGKIA)₃-NH₂ | 8 | 8 | 32 | 64 | 9 |
| (KLAGLAK)₃-NH₂ | 8 | 16 | 128 | 64 | 9 |
| (KIGAKI)₃-NH₂ | 8 | 16 | 32 | 64 | 3 |
| A₁₉-Magainin 2-NH₂ | 8 | 64 | 16 | 128 | 29 |
| MSI-78 | 8 | 8 | 4 | 64 | 9 |

^{a}See Table 1 for peptide sequences.
^{b}For minimum inhibitory concentrations, differences of greater than a factor of two are considered significant.

TABLE 3
Effect of Peptides on Bilayer-to-Hexagonal Phase
Transition (T_{H}) of DiPoPE

| Peptide | Linear Regression^{a} |
|---|---|
| KIAGKIAx3 | 132 ± 65 |
| KLAGLAKx3 | 1,077 ± 52 |
| KIGAKIx3 | 157 ± 142 |

^{a}Slope of TH vs. mole fraction of peptide determined by regression analysis.

TABLE 4

| | | | | | | | MIC (μg/mL) | | |
|---|---|---|---|---|---|---|---|---|---|
| | | | | | μ | μ | | Staph. | Pseud. |
| Peptide | Size | MW | +n | H | (alpha) | (beta) | E. coli | aureus | aerug |
| KIAGKIAx3 | 21 | 2070 | +7 | −0.011 | 0.399 | 0.158 | 8 | 8 | 32 |
| KL-14 | 14 | 1788 | +8 | −0.296 | 0.040 | 0.804 | 32 | 16 | 16 |
| KL-18 | 18 | 2273 | +10 | −0.294 | 0.009 | 0.806 | 64 | 16 | 32 |
| KIGAKIx3 | 18 | 1930 | +7 | −0.075 | 0.020 | 0.605 | 8 | 8 | 32 |

TABLE 102

| | | | | | | | MIC (μg/mL) | | |
|---|---|---|---|---|---|---|---|---|---|
| | | | | | μ | μ | | Staph. | Pseud. |
| Peptide | Size | MW | +n | H | (alpha) | (beta) | E. coli | aureus | aerug |
| 1,2-Hex | 18 | 1930 | +7 | −0.075 | 0.020 | 0.005 | 128 | >256 | 128 |
| 1,3-Hex | 18 | 1930 | +7 | −0.075 | 0.020 | 0.575 | 8 | 32 | 16 |
| 1,4-Hex | 18 | 1930 | +7 | −0.075 | 0.020 | 0.155 | 32 | >256 | 64 |
| 1,5-Hex | 18 | 1930 | +7 | −0.075 | 0.020 | 0.605 | 8 | 16 | 16 |
| 1,6-Hex | 18 | 1930 | +7 | −0.075 | 0.020 | 0.005 | >256 | >256 | >256 |
| 1,2-Hept | 21 | 2143 | +7 | −0.076 | 0.281 | 0.015 | 8 | 16 | 16 |
| 1,3-Hept | 21 | 2143 | +7 | −0.076 | 0.131 | 0.155 | 32 | 256 | >256 |
| 1,4-Hept | 21 | 2143 | +7 | −0.076 | 0.390 | 0.027 | 8 | 8-16 | 32 |
| 1,5-Hept | 21 | 2143 | +7 | −0.076 | 0.375 | 0.147 | 8 | 4 | 16 |
| 1,6-Hept | 21 | 2143 | +7 | −0.076 | 0.055 | 0.009 | >256 | >256 | >256 |
| 1,7-Hept | 21 | 2143 | +7 | −0.076 | 0.246 | 0.120 | 8 | 16 | 16 |

TABLE 103

| | | | | | | | MIC (μg/mL) | | |
|---|---|---|---|---|---|---|---|---|---|
| | | | | | μ | μ | | Staph. | Pseud. |
| Peptide | Size | MW | +n | H | (alpha) | (beta) | E. coli | aureus | aerug |
| KIGAKI | 18 | 1930 | +7 | −0.075 | 0.020 | 0.605 | 8 | 8 | 32 |
| Oct-KIGAKI-18A | 18 | 2055 | +6 | −0.075 | 0.020 | 0.605 | 8 | 4 | 8 |
| Oct-KIGAKI-18B | 18 | 2055 | +6 | −0.075 | 0.020 | 0.605 | 8 | 8 | 8 |
| Oct-KIGAKI-12 | 12 | 1442 | +4 | −0.085 | 0.244 | 0.600 | | | |
| Oct-KIGAKI-6 | 6 | 829 | +2 | −0.155 | 0.433 | 0.565 | >256 | >256 | >256 |
| Oct-Beta-11 | 11 | 1557 | +6 | −0.374 | 0.133 | 0.826 | 16 | 4 | 8 |
| KL-14 | 14 | 1788 | +8 | −0.296 | 0.040 | 0.804 | 32 | 16 | 16 |
| KL-18 | 18 | 2273 | +10 | −0.294 | 0.009 | 0.806 | 64 | 16 | 32 |

1. An antimicrobial compound which is (a) a peptide having a length of 8-50 amino acids, a net charge of at least four, a hydrophobic moment as a beta sheet which is at least 0.2 higher than its hydrophobic moment as an alpha helix, and having detectable membrane disrupting activity against at least one microbial pathogen, and substantially no membrane disrupting activity against mammalian cells, or (b) a peptoid, peptidomimetic or nonpeptidic analogue of a peptide according to (a) above.

2. The compound of claim 1 which is a peptide.

3. The compound of claim 1 which is a peptide characterized by replacement of the C-terminal-COOH with-NH2.

4. The compound of claim 2 which is a peptide characterized by replacement of the N-terminal NH2— with an alkanoyl group of 2 to 12 carbons.

5. The compound of claim 2 wherein the peptide consists of genetically encoded amino acids.

6. The compound of claim 2 which has a net charge of at least +5.

7. The compound of claim 2 which has a net charge of at least +6.

8. The compound of claim 2 which has a net charge of at least +7.

9. The compound of claim 2 which has a net charge which is not more than +10.

10. The compound of claim 2 which has a charge density in the range of 0.25 to 0.5 charges per total length in amino acids.

11. The compound of claim 2 which has a charge density in the range of 0.075 to 0.15 charges per length of compound in angstroms.

12. The compound of claim 2 which has an arithmetic mean hydrophobicity which is at least −0.8, as measured on Eisenberg's non-normalized consensus hydrophobicity scale.

13. The compound of claim 2 which has an arithmetic mean hydrophobicity which is at least −0.6, as measured on Eisenberg's non-normalized consensus hydrophobicity scale.

14. The compound of claim 1 which is a peptide (or a mimetic of a peptide) of not more than 30 amino acids.

15. The compound of claim 1 which is a peptide (or a mimetic of a peptide) of not more than 20 amino acids.

16. The compound of claim 2 which, when bound to a lipid vesicle model of a bacterial membrane, is at least 50% in the beta sheet conformation.

17. The compound of claim 2 which, when bound to a lipid vesicle model of a bacterial membrane, is at least 80% in the beta sheet conformation.

18. The compound of claim 2 which, when bound to a lipid vesicle model of a bacterial membrane, is at least 90% in the beta sheet conformation.

19. The compound of claim 2 which, when bound to a lipid vesicle model of a bacterial membrane, is at least 95% in the beta sheet conformation.

20. The compound of claim 1 which has an alpha moment of less than 0.2.

21. The compound of claim 1 which has an alpha moment of less than 0.1.

22. The compound of claim 1 which has an alpha moment of less than 0.05.

23. The compound of claim 1 which has a beta moment greater than 0.2.

24. The compound of claim 1 which has a beta moment greater than 0.4.

25. The compound of claim 1 which is perfectly amphipathic as a beta strand.

26. The compound of claim 1 which is nearly perfectly amphipathic as a beta strand.

27. The compound of claim 1 which is of the form (KIGAKI)ₙ where n is 1 to 8.

28. The compound of claim 1 which is of the form (KL)ₙ or (KI)ₙ, where n is 4 to 25.

29. A method of inhibiting microbial activity in a mammal which comprises administering to a mammal an inhibitory amount of an antimicrobial compound according to claim 1.

30. The method of claim 29 in which the mammal is a human.

31. A method of inhibiting nonmicrobial pathogenic activity in a mammal which comprises administering to a mammal an inhibitory amount of a compound according to claim 1 which has detectable membrane disrupting activity against said pathogen.

32. A method of killing human sperm which comprises contacting the sperm with a lethal amount of a compound according to claim 1 which has detectable membrane disrupting activity against human sperm.
